SEQUENCE LISTING (1 sequence)

SEQ ID NO: 1 - DNA, 82 bp, Artificial Sequence
  Synthetic
  misc_feature (31)..(44): n is a, c, g, or t
ttttttttttttttttttttttttttttttnnnnnnnnnnnnnncttctccatgagacgcaactatggtgacgaattttttt

(19) United States
(12) Patent Application Publication
(10) Pub. No.: US 2021/0024919 A1
(43) Pub. Date: Jan. 28, 2021

(54) FUNCTIONALIZED SOLID SUPPORT

(71) Applicant: Massachusetts Institute of Technology, Cambridge, MA (US)

(72) Inventors: Alexander K. Shalek, Cambridge, MA (US); Andrew Navia, Cambridge, MA (US); Benjamin E. Mead, Cambridge, MA (US)

(21) Appl. No.: 16/936,197

(22) Filed: Jul. 22, 2020

Related U.S. Application Data
(60) Provisional application No. 62/876,909, filed on Jul. 22, 2019.

(51) Int. Cl. C12N 15/10; C12Q 1/6869; B01J 19/00

(52) U.S. Cl. CPC C12N 15/1065; C12Q 1/6869; B01J 19/0046; B01J 2219/005; B01J 2219/00693; B01J 2219/00576; B01J 2219/00722; B01J 2219/00547

(57) ABSTRACT

The present invention relates to functionalized solid supports and methods of making functionalized solid supports. Methods for preparing a population of high quality functionalized solid supports for use in various nucleic acid analysis methods are provided. The invention also provides methods for validation and quality control of the functionalization steps.

### CROSS-REFERENCE TO RELATED APPLICATIONS

This application claims the benefit of U.S. Provisional Application No. 62/876,909, filed Jul. 22, 2019. The entire contents of the above-identified applications are hereby fully incorporated herein by reference.

### REFERENCE TO AN ELECTRONIC SEQUENCE LISTING

The contents of the electronic sequence listing (BROD_4330US_ST25.txt“; Size is 4 Kilobytes and it was created on Jul. 21, 2020) is herein incorporated by reference in its entirety.

### TECHNICAL FIELD

The subject matter disclosed herein is generally directed to functionalized solid supports and methods for preparing and assaying the solid support.

### BACKGROUND

Capture beads have been extensively used in single cell sequencing methods. RNA-sequencing methods are costly, and single or few screen readouts leaves open the possibility for false-positives in phenotypic screening, particularly so in organoids. Artifacts of assays can drive misinterpretation. The efficiency of the methods depend, in part, on the quality and coupling efficiency of the beads. A major limitation of commercially-available capture beads include batch-to-batch variability in quality, i.e., the quality of the beads is variable and inconsistent. This can include the number of oligonucleotide strands on each bead, if any, and the quality of the oligonucleotide strands and level of degradation in the synthesis process. In addition, current quality control (QC) protocols, if any, fail to robustly characterize and validate synthesis batches. Quality control of solid-state beads constitutes a challenge, in part, due to the limited methods of analyses for the solid-state products.

To overcome these limitations, Applicants have developed methods to improve the syntheses of the beads and novel quality control methods to robustly characterize and validate the syntheses of capture beads to ensure that the quality of the beads is consistent and that the beads produced are of a high-quality product. The quality of the beads provides Applicants the ability to confirm oligo sequence in a more efficient manner.

### SUMMARY

In certain example embodiments, methods for preparing a population of functionalized solid support are provided comprising surface reactive nucleic acid molecules in sequence(s). In particular embodiments, the methods comprise the step of reacting a solid support comprising a surface reactive nucleic acid molecule with another nucleic acid molecule so as to obtain a solid support comprising surface reactive nucleic acid molecules in sequence(s). In one aspect, the reacting with another nucleic acid molecule comprises reacting with a dinucleotide or a trinucleotide. In an aspect, the reacting step is performed n times, wherein n is an integer between 1 and 150.

The reacting step can be repeated so as to obtain surface reactive nucleic acid molecules in sequences of a Universal sequence, barcode, a Unique Molecular Identifier (UMI) and a capture sequence. The reacting step can be repeated to as to obtain surface reactive nucleic acid molecules comprising one or more of: oligonucleotides, nucleotides, analogs thereof, a molecular barcode, a Unique Molecular Identifier, a oligodT, an amplification primer, a cell type specific sequence, a pathogen-specific sequence, a TCR or BCR specific sequence, or primers for specific genes or pools of genes. In certain embodiments, the cell type specific sequence comprises mutation specific sequences, sequences adjacent to a feature of interest, gene pools.

In embodiments, one or more of the nucleic acid molecule(s) comprise a protecting group. In one aspect, after the reacting step, deprotecting of the nucleic acid molecules is performed at room temperature in ammonia hydroxide, optionally 30% ammonia hydroxide for one hour.

The solid support can comprise a bead, micro-bead, micro-assay, micro-well, or micro-lid. In an aspect, the solid support comprises a bead that is a silica bead, a hydrogel bead or a magnetic bead. In embodiments, the solid support comprises a magnetic core. In one aspect, the solid support has an average particle size between about 10 microns to 200 microns, about 10 microns to 30 microns, about 30 microns to 50 microns, about 50 microns to 100 microns, about 100 microns to 200 microns, or about 30 microns.

In embodiments, the solid support comprises a polymer, optionally a hydroxylated methacrylic polymer, a hydroxylated poly(methyl methacrylate), a polystyrene polymer, a polypropylene polymer, a polyethylene polymer agarose, or cellulose. In some embodiments, the solid support comprises a hydrogel bead or a magnetic bead.

In embodiments, the functionalized solid support comprises a spacer. The spacer having a functional group exposed for reaction is prepared by a method including the steps of reacting a solid support having surface bearing reacting groups with an activator, so as to obtain a solid support with an activated surface comprising an activating moiety, and reacting the activated surface with a spacer compound having a first moiety that reacts with the activating moiety and optionally a second moiety comprising a functional group. In an aspect, the functional group exposed for reaction comprises an amine, hydroxyl, carboxyl, thiol.

In certain embodiments, the solid support comprises a spacer, the spacer optionally comprising a polyethylene glycol polymer (PEG), alkyl amine, or polysaccharide linker. In an aspect, the PEG is a hetero-functional PEG comprising two or more different functionalities, wherein at least one of the functionalities is a primary amine, hydroxyl, thiol, methoxy, or other capping group. In some embodiments, the PEG has a molecular weight range of about 2,000 daltons to 10,000 daltons, a molecular weight of about 2000 Daltons, 3500 Daltons, 5000 Daltons, 8000 Daltons, 9000 Daltons, or about 10,000 Daltons. In embodiments, the spacer comprises a photolabile linker, a fluoride ion labile linker, or a cleavable linker. The spacer can comprise a benzenesulfonylethyl linker, an o-nitrobenzyl carbonate photolabile linker, a 5-methoxy-2-nitrobenzyl carbonate photolabile linker, an o-nitrophenyl-1,3-propanediol base photolabile linker, a fluoride ion labile diisopropylsilyl linker, a fluoride ion labile disiloxyl phosphoramidite linker, a NPE carbonate linker, a 9-fluorenylmethyl linker, a phthaloyl linker, an oxalyl linker, a malonic acid linker, a diglycolic acid linker, a hydroquinone-O,O′-diacetic acid (Q-linker), or a thiophospate linker. In particular embodiments, the spacer comprises a benzenesulfonylethyl linker cleavable with triethylamine/dioxane, a nonyl phenol ethoxylate (NPE) carbonate linker cleavable with 1,8-Diazabicyclo[5.4.0]undec-7-ene (DBU)/pyridine, or a 9-fluorenylmethyl linker or a phthaloyl linker cleavable with DBU. The spacer can comprise a succinic acid linked to an N-methylglycine (sarcosine) derivatized support, a succinic acid linked to 1,6-bis methylaminohexane spacer, a succinic acid linked to N-propyl polyethylene glycol Tentagel support, or a succinyl-sarcosine linkage.

In certain embodiments, the nucleic acid molecules are in a 3′ to 5′ orientation, or wherein the nucleic acid molecules are in a 5′ to 3′ orientation.

Methods of preparing a plurality of solid supports are provided. In particular embodiments, the solid supports each comprise a well barcode, primers, a unique molecular identifier, and a capture oligonucleotide, which may be an oligo-dT. In certain embodiments, the methods further comprise pooling beads in an individual discrete volume, seeding cells in the individual discrete volume, and conducting high throughput RNA sequencing for a population of cells in each individual discrete volume. In particular embodiments, the well barcode comprises about 6 nucleotides; in certain embodiments, the UMI comprises about 14 oligonucleotides. In particular methods, the method further comprises compressive sensing for ultra-low depth sequencing.

Methods of detecting the presence of functionalization on a solid support are provided and comprise the steps of contacting the solid support with a fluorescent probe, the fluorescent probe comprising an oligonucleotide, the oligonucleotide capable of binding moieties when present on the solid support. The method can further comprise a step of quantifying the amount of functionalization of the solid support. In an aspect, quantifying comprises measuring the level of fluorescence, the fluorescence correlating to the amount of functionalization. Steps of detecting the presence of functionalization can further comprise sorting the solid support fluorescently. In an aspect, the method can further comprise removing the probe subsequent to sorting.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A—Efficient Photocleavable sequences on beads: This probe bound the PCR region of the bead. Here Applicants are showing the loss of oligos after applying a UV light to the Phot—cleavable linker.

FIG. 1B—This probe bound the PCR region of the bead. Here, Applicants are showing the loss of oligos after applying a UV light to the Phot—cleavable linker. The bottom two plots show Applicants' most recent batches and release more oligos than the chemgenes beads (top left).

FIG. 1C—The bottom two plots show Applicants' most recent batches and release more oligos than the commercially available chemgenes beads (top left).

FIG. 2—An overview of Applicants' proposed probe use, with probes that can bind any consistent sequence on the bead.

FIG. 3—Chart showing probe binds specifically to its complement sequence and will not bind unprocessed oligonucleotides. The lines are beads with randomer sequences. Applicants' probe does not bind those oligos. The far left line is beads that were not deprotected; the probe does not bind those sequences, and the far right line is binding the PCR handle of Applicants' beads.

FIG. 4—presently disclosed probes can distinguish high poly-T content/bead functionalization from beads with little to no functionalization. These beads had a probe that bound the poly-A capture region. Gate E is the same for both sorts and is based on where the majority of beads sit. Gates F and G are meant to show what percentage of beads have “a lot” of oligos, excluding beads that fall below 10̂2. It is unclear at what point on the x-axis beads no longer become viable. Gate F for Applicants' beads contain a higher percentage of beads than gate G of the chemgenes comparative commercial beads.

FIG. 5—Effect of heat versus no heat for functionalization of beads; specificity of FISH probe of comparative commercial bead and presently disclosed beads.

FIG. 6—Exemplary design of population beads.

FIG. 7—pilot round 1 wherein lysates frozen and added to well-coded beads.

FIG. 8—WTA PCR, Nextera PCR comparison shows WTA PCR of 9 cycles and Nextera PCR of 12 cycles. NExtSeq loaded @ 2.2 pM. WTA+Nextera+NextSeq−force cell count+feed in specified barcodes for alignment.

FIG. 9—alignment with DropSeq pipeline, with adjustment of barcode and UMI length with bar chart measuring alignment quality and graph providing number of reads per cell barcode at 20k cells, 10k cells, 5k cells and 1k cells.

FIG. 10—nUMI is independent of cell number. Violin plots of identity vs. expression level at each of cell seedings 1k, 5k, 10k, 20k (upper panel) and plot of nCount RNA versus ATP

FIG. 11 includes a graph of UMI rank averaged over all cell seedlings.

FIG. 12A plots sample UMIs greater than 5k by cell type; FIG. 12B includes a heat map of gene expression by cell type.

FIGS. 13A-13B—FIG. 13A A Klenow extension can create the final capture bead; FIG. 13B depicts use of DNA probe binding to capture bead for quality control evaluation.

FIG. 14 Fluorescently-activated cell sorting (FACS) analysis of oligo density based on distinct size gates

FIG. 15 shows samples using varied extension conditions, including temperature of annealing and time at temperature

FIG. 16 includes evaluation of extension reaction optimization, showing slight effect of extension handle concentration

FIGS. 17A-17B Single cell data isolating higher quality beads using probe system (17A) commercially available beads (17B) initial sort of Applicants beads on left, Applicants beads sorted after 6-7 hours, showing probe shedding

FIGS. 18A-18C Histograms of Transcript (left) and Gene (right) count from gated analysis including the top gate (18A) no sort (18B) and smaller gate (18C).

FIG. 19 is a histogram of beads using quality control methods, and showing the most populated barcodes were identical, indicating a split-pool issue.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011)

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

Reference is made to U.S. Provisional Application No. 62/575,883, filed Oct. 23, 2017 and International Patent Application No. PCT/US2013/060990 filed Mar. 13, 2013, and International Patent Publication No. WO 2019/084058 filed Oct. 23, 2018 incorporated by reference herein in their entirety.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide improved methods for preparing a population of functionalized solid supports. The functionalized solid supports comprising surface reactive nucleic acid molecules in sequences are prepared comprising reacting a solid support comprising a surface reactive nucleic acid molecule with another nucleic acid molecule so as to obtain a solid support comprising surface reactive nucleic acid molecules in sequence(s). Methods disclosed herein can be utilized to prepare the solid support, which can be part of a library of unique labels or barcodes. Such libraries may be of any size, and are preferably large libraries including hundreds of thousands to billions of unique labels.

The methods developed by Applicants provide increases in the quality of the individual beads, i.e., higher percentage of functionalization, increase in the number of reactive sites, higher oligo density, higher transcript capture efficiency, increase in oligo sequence consistency in terms of identity and length of sequences, and monodisperity, i.e., particle size uniformity.

Methods of detecting the presence of functionalization on a solid support are also provided. Detecting the presence of functionalization on a solid support can include the steps of contacting the solid support with a fluorescent probe, the fluorescent probe comprising an oligonucleotide, the oligonucleotide capable of binding moieties when present on the solid support.

### Functionalized Solid Support

In an embodiment, the present invention provides a solid support that has been functionalized to comprise one or more agents.

A solid support can mean a bead or micro-bead, or a plurality of micro-beads, micro-arrays, micro-wells, or micro-lids. The solid support can be shaped in any manner required for an end use application and may have a shape that is circular, square, star, or porous. Examples of suitable solid supports include, but are not limited to, inert polymers (preferably non-nucleic acid polymers), beads, glass, or peptides. In some embodiments, the solid support is an inert polymer or a bead. The bead is a silica bead, a hydrogel bead or a magnetic bead. In some embodiments, the solid support comprises a magnetic core. In certain embodiments, when using magnetic core, photocleavable linkers can be utilized with a terminal phosphate group for ligation.

Examples of suitable polymers include a hydroxylated methacrylic polymer, a hydroxylated poly(methyl methacrylate), a polystyrene polymer, a polypropylene polymer, a polyethylene polymer agarose, or cellulose.

The solid support may be functionalized to permit covalent attachment of the agent and/or label. Such functionalization on the support may comprise reactive groups that permit covalent attachment to an agent and/or a label.

In particular embodiments, the solid support has an average particle size between about 10 microns to 200 microns, about 10 microns to 190 microns, about 10 microns to 180 microns, about 10 microns to 170 microns, about 10 microns to 160 microns, about 10 microns to 150 microns, about 10 microns to about 140 microns, about 10 to about 130 microns, about 10 to about 120 microns, about 10 microns to about 110 microns, about 10 microns to about 100 microns, about 10 microns to about 90 microns, about 10 microns to about 80 microns, about 10 microns to about 70 microns, about 10 microns to about 60 microns, about 10 microns to about 50 microns, about 10 microns to about 40 microns, about 10 microns to 30 microns, about 10 microns to about 20 microns, about 20 microns to about 30 microns, about 20 microns to about 40 microns, about 20 microns to about 50 microns, about 20 microns to about 60 microns, about 20 microns to about 70 microns, about 20 microns to about 80 microns, about 20 microns to about 100 microns, about 20 microns to about 100 microns, about 50 microns to about 100 microns, about 100 microns to 200 microns, or about 30 microns. In some embodiments, the bead or micro-bead has an average size, measured as average diameter of 20-40 μm.

### Spacers

A spacer as used herein, may comprise a polyethylene glycol polymer (PEG), a polysaccharide, an alkyl amine or a linker. Spacers can be optionally included in the solid supports. In some embodiments, the spacer further comprises a linker. Spacer link may vary according to the size of the functional support.

In particular embodiments, the spacer is preferably a PEG spacer or non-PEG spacer, e.g., long chain alkyl amines (LCAA) (see also Guzaev, Andrei, “Solid-Phase Supports for Oligonucleotide Synthesis,” Current Protocols in Nucleic Acid Chemistry (2010) 3.1.1-3.1.28), and the spacer can be a linker or comprise a linker.

In addition, a spacer can include linker arms that allow on-column deprotection and then optional cleave. For example, succinic acid linked to an N-methylglycine (sarcosine) derivatized support (Brown, T., Pritchard, C. E., Turner, G., and Salisbury, S. A. 1989. A new base-stable linker for solid-phase oligonucleotide synthesis. J. Chem. Soc. Chem. Commun. 891-893.), succinic acid linked to 1,6-bis methylaminohexane spacer (Stengele, K. P. and Pfleiderer, W. 1990. Improved synthesis of oligodeoxyribonucleotides. Tetra-hedron Lett. 31:2549-2552.), succinic acid linked to N-propyl polyethylene glycol Tentagel support (Weiler, J. and Pfleiderer, W. 1995. An improved method for the large-scale synthesis of oligonucleotides applying the NPE/NPEOC strategy. Nucleos. Nucleot. 14:917-920.), succinyl-sarcosine linkage for the solid-phase synthesis of branched oligonucleotides (Grotli, M., Eritja, R., and Sproat, B. 1997. Solid phase synthesis of branched RNA and branched DNA/RNA chimeras. Tetrahedron 53:11317-11347.), linkage through the amino group of cytosine for branched and cyclic oligonucleotide synthesis (De Napoli, L., Galeone, A., Mayol, L., Messere, A., Montesarchio, D., and Piccialli, G. 1995. Auto- mated solid phase synthesis of cyclic oligonu- cleotides: A further improvement. Bioorgan. Med. Chem. 3:1325-1329.), Oxidizable solid support (Bower, M., Summers, M. F., Kell, B., Hoskins, J., Zon, G., and Wilson, W. D. 1987. Synthesis and characterization of oligodeoxyribonucleotides containing terminal phosphates. NMR, UV spectroscopic and thermodynamic analysis of duplex formation of [d(pGGATTCC)]₂ (SEQ ID NO:1), [d(GGAAT- TCCp)]₂ (SEQ ID NO:2) and [d(pGGAATTCCp)]₂ (SEQ ID NO:3). Nucl. Acids Res. 15:3531-3547.; Markiewicz, W. T., Adrych-Rozek, K., Markiewicz, M., Zebrowska, A., and Astriab, A. 1994. Synthesis of oligonucleotides permanently linked with solid supports for use as synthetic oligonucleotide combinatorial libraries. In Innovation and Perspectives in Solid Phase Synthesis: Peptides, Proteins and Nucleic Acids: Biological and Biomedical Applications (R. Epton, ed.) pp. 339-346. Mayflower Worldwide, Birmingham.), Phenyl thioether linker, which is stable until oxidized into a phenylsulfone (Felder, E., Schwyzer, R., Charubala, R., Pfleiderer, W., and Schulz, B. 1984. A new solid phase approach for rapid synthesis of oligonucleotides bearing a 3′-terminal phosphate group. Tetrahedron Lett. 25:3967-3970.), Thiophosphate linker, cleavable by iodine/water oxidation or acetic acid hydrolysis (Tanaka, T., Yamada, Y Uesugi, S., and Ikehara, M. 1989. Preparation of a new phosphorylating agent: S-(N-monomethoxytritylaminoethyl)-O- (o-chlorophenyl)phosphorothioate and its application in oligonucleotide synthesis. Tetrahedron 45:651-660.), 3-Chloro-4-hydroxyphenyl linker for the solid-phase synthesis of cyclic oligonucleotides (Alazzouzi, E., Escaja, N., Grandas, A., and Pedroso, E. 1997. A straightforward solid-phase synthesis of cyclic oligodeoxyribonucleotides. Angew. Chem. Intl. Ed. Engl. 36:1506-1508.), Linker arm produced from tolylene 2,6-diisocyanate with more stable carbamate and urethane linkages (Kumar, A. 1994. Development of a suitable linkage for oligonucleotide synthesis and preliminary hybridization studies on oligonucleotides synthesized in situ. Nucleos. Nucleot. 13:2125-2134.; Sproat, B. S. and Brown, D. M. 1985. A new linkage for solid phase synthesis of oligodeoxyribonucleotides. Nucl. Acids Res. 13:2979-2987.). A spacer can also include linker arms for permanent attachment to solid-phase supports, e.g., Hydroxy propylamine linker (Seliger, H., Bader, R., Birch-Hirschfield, E., Föides- Papp, Z., Hinz, M., and Scharpf, C. 1995. Surface reactive polymers for special applications in nucleic acid synthesis. Reactive Functional Polymers 26:119-126.), Dimethoxytrityl glycolic acid linker (Hakala, H., Heinonen, P., Iitia, A., and Lonnberg, H. 1997. Detection of oligonucleotide hybridization on a single microparticle by time-resolved fluorometry: Hybridization assays on polymer particles obtained by direct solid phase assembly of the oligonucleotide probes. Bioconjugate Chem. 8:378-384.), Dimethoxytrityl-4, 7, 10,13 -tetraoxatridecanoate linker (Markiewicz, W. T., Adrych-Rozek, K., Markiewicz, M., Zebrowska, A., and Astriab, A. 1994. Synthesis of oligonucleotides permanently linked with solid supports for use as synthetic oligonucleotide combinatorial libraries. In Innovation and Perspectives in Solid Phase Synthesis: Peptides, Proteins and Nucleic Acids: Biological and Biomedical Applications (R. Epton, ed.) pp. 339-346. Mayflower Worldwide, Birmingham.), Long spacer linkages prepared using repetitive coupling of various phosphoramidites (Shchepinov, M. S., CaseGreen, S. C., and Southern, E. M. 1997. Steric factors influencing hybridisation of nucleic acids to oligonucleotide arrays. Nucl. Acids Res. 25:1155-1161.), Cleavable spacer linkage used in conjunction with the preceding phosphoramidites to control the surface oligonucleotide density (Shchepinov, M. S., CaseGreen, S. C., and Southern, E. M. 1997. Steric factors influencing hybridisation of nucleic acids to oligonucleotide arrays. Nucl. Acids Res. 25:1155-1161.), Direct phosphate linkage to surface silanol groups (Cohen, G., Deutsch, J., Fineberg, J., and Levine, A. 1997. Covalent attachment of DNA oligonucleotides to glass. Nucl. Acids Res. 25:911-912.), Diol linker formed from 3-glycidoxypropyl trimethoxysilane (Maskos, U. and Southern, E. M. 1992. Oligonucleotide hybridizations on glass supports: A novel linker for oligonucleotide synthesis and hybridisation properties of oligonucleotides synthesized in situ. Nucl. Acids Res. 20:1679-1684.), Polyethylene glycol linkers (Maskos, U. and Southern, E. M. 1992. Oligonucleotide hybridizations on glass supports: A novel linker for oligonucleotide synthesis and hybridisation properties of oligonucleotides synthesized in situ. Nucl. Acids Res. 20:1679-1684.), Bis-(2-hydroxethyl)-aminopropylsilane linker with hexaethylene glycol spacer phosphoramidites (Pease, A. C., Solas, D., Sullivan, E. J., Cronin, M. T., Holmes, C. P., and Fodor, S. P. A. 1994. Light- generated oligonucleotide arrays for rapid DNA sequence analysis. Proc. Natl. Acad. Sci. U.S.A. 91:5022-5026.), N-(3 -(triethoxysilyl)-propyl)-4-hydroxybutyrami de linker (McGall, G. H., Barone, A. D., Diggelmann, M., Fo-dor, S. P. A., Gentalen, E., and Ngo, N. 1997. The efficiency of light-directed synthesis of DNA arrays on glass substrates. J. Am. Chem. Soc. 119:5081-5090.), Linkage through the N⁴-position of cytosine (Markiewicz, W. T., Adrych-Rozek, K., Markiewicz, M., Zebrowska, A., and Astriab, A. 1994. Synthesis of oligonucleotides permanently linked with solid supports for use as synthetic oligonucleotide combinatorial libraries. In Innovation and Perspectives in Solid Phase Synthesis: Peptides, Proteins and Nucleic Acids: Biological and Biomedical Applications (R. Epton, ed.) pp. 339-346. Mayflower Worldwide, Birmingham.), Triethylene glycol ethylacrylamide linker (Markiewicz, W. T., Adrych-Rozek, K., Markiewicz, M., Zebrowska, A., and Astriab, A. 1994. Synthesis of oligonucleotides permanently linked with solid supports for use as synthetic oligonucleotide combinatorial libraries. In Innovation and Perspectives in Solid Phase Synthesis: Peptides, Proteins and Nucleic Acids: Biological and Biomedical Applications (R. Epton, ed.) pp. 339-346. Mayflower Worldwide, Birmingham.).

In a preferred embodiment, the linker is photocleavable. In embodiments, the photocleavable linker comprises DMTO. Suitable photocleavable linkers and spacers are available, for example, at Glen Research. Two suitable photocleavable linkers from Glen Research include, for example, PC linker and PC spacer.

A spacer is preferably multifunctional, in some embodiments bifunctional, and can be either homo-functional or hetero-functional. For example, a PEG spacer can have an amine on one end and a thiol on the other end of the molecule, or amine and hydroxyl heterobifunctional PEGs. The functionality of the spacer may be capped with protecting groups, for example methoxy groups. Depending on the number of functional groups on the beads, which varies based on bead size and material, some functional groups can be blocked, for example, with a methoxy PEG to block some of the functional groups, which can improve performance of the solid support.

Examples of hydroxyl protecting groups include e.g., 3-nitro-2-pyridinesulfenyl (Npys); dimethoxytrityl (DMT); monomethyoxytrityl (MMT); Acetyl; benzyl; Benzoyl; Beta-methoxyethoxymethyl ether; Methoxymethyl ether; p-methyoxybenzyl ether; methyl-thiol methyl ether; Pivaloyl (Piv); Tetrahydropyranyl (THP); Tetrahydrofuran (THF); Trityl; Silyl ether; Methyl ether; and Ethoxy ethyl ethers. Examples of amine protecting groups include, e.g., Carbobenzyloxy; p-Methoxybenzylcarbonyl; tert-Butyloxycarbonyl; 9-fluorenylmethyloxycarbonyl (FMOC); Acetyl; Benzoyl; Benzyl; Carbamate; p-Methyoxybenzyl; 3,4-dimethoxybenzyl; p-methoxyphenyl; Tosyl; TROC; NO SL; and NP S. Examples of carboxylic acid protecting groups include, e.g., Methyl Esters; Benzyl Esters; Tert-butyl esters; Esters of 2,6 disubstituted phenyls; Silyl esters; Orthoesters; and Oxazoline. Examples of phosphate protecting groups include, e.g., 2-cyanoethyl and methyl. Examples of terminal alkyne protecting groups include, e.g., propargyl alcohols and silyl groups.

Protecting groups of 3′ terminal hydroxyl groups of surface reactive nucleotides can be protected, for example with DMT. In some embodiments, a nucleoside phosphoramidite can be coupled to surface reactive nucleic acid and creating a phosphite triester. The unreacted 3′-hydroxyl groups are than capped with a protecting group, e.g., acetic anhydride and N-methylimidazole. The phosphate triester is then oxidized.

In another example, a label can be attached to an agent via a linker or in another indirect manner. Examples of linkers, include, but are not limited to, carbon-containing chains, polyethylene glycol (PEG), nucleic acids, monosaccharide units, and peptides. The linkers may be cleavable under certain conditions.

Cleavable linkers are known in the art and include, but are not limited to, TEV, trypsin, thrombin, cathepsin B, cathespin D, cathepsin K, caspase 1,matrix metalloproteinase sequences, phosphodiester, phospholipid, ester, β-galactose, dialkyl dialkoxysilane, cyanoethyl group, sulfone, ethylene glycolyl disuccinate, 2-N-acyl nitrobenzenesulfonamide, a-thiophenylester, unsaturated vinyl sulfide, sulfonamide after activation, malondialdehyde (MDA)-indole derivative, levulinoyl ester, hydrazone, acylhydrazone, alkyl thioester, disulfide bridges, azo compounds, 2-Nitrobenzyl derivatives, phenacyl ester, 8-quinolinyl benzenesulfonate, coumarin, phosphotriester, bis-arylhydrazone, bimane bi-thiopropionic acidderivative, paramethoxybenzyl derivative, tert-butylcarbamate analogue, dialkyl or diaryl dialkoxysilane, orthoester, acetal, aconityl, hydrazone, b-thiopropionate, phosphoramidate, imine, trityl, vinyl ether, polyketal, alkyl 2-(diphenylphosphino)benzoate derivatives, allyl ester, 8-hydroxyquinoline ester, picolinate ester, vicinal diols, and selenium compounds (see, e.g. Leriche G, Chisholm L, Wagner A. Cleavable Linkers in Chemical Biology. Bioorg Med Chem. 15;20(2):571-82. 2012, which is incorporated herein by reference). Cleavage conditions and reagents include, but are not limited to, enzymes, nucleophilic/basic reagents, reducing agents, photo-irradiation, electrophilic/acidic reagents, organometallic and metal reagents, and oxidizing reagents.

Examples of linkers include a succinyl linker, o-Nitrobenzyl carbonate photolabile linker arm (Greenberg, M. M. and Gilmore, J. L. 1994. Cleavage of oligonucleotides from solid-phase supports using O-nitrobenzyl photochemistry. J. Org. Chem. 59:746-753.), 5-Methoxy-2-nitrobenzyl carbonate photolabile linker arms (Venkatesan, H. and Greenberg, M. M. 1996. Improved utility of photolabile solid phase synthesis supports for the synthesis of oligonucleotides containing 3′-hydroxyl termini. J. Org. Chem. 61:525-529.), o-Nitrophenyl-1,3-propanediol base photolabile linker for 3′-phosphorylated oligonucleotides (Dell' Aquila, C., Imbach, J. L., and Rayner, B. 1997. Photolabile linker for the solid-phase synthesis of base-sensitive oligonucleotides. Tetrahedron Lett. 38:5289-5292.), Fluoride ion labile diisopropylsilyl linker arm (Routledge, A., Wallis, M. P., Ross, K. C., and Fraser, W. 1995. A new deprotection strategy for automated oligonucleotide synthesis using a novel silyl-linked solid support. Bioorgan. Med. Chem. Lett. 5:2059-2064.), Fluoride ion labile disiloxyl phosphoramidite linker arm (Kwiatkowski, M., Nilsson, M., and Landegren, U. 1996. Synthesis of full-length oligonucleotides: Cleavage of apurinic molecules on a novel sup-port. Nucl. Acids Res. 24:4632-4638.), Benzenesulfonylethyl linker arm cleavable with triethylamine/dioxane (Efimov, V. A., Buryakova, A. A., Reverdatto, S. V., Chakhmakhcheva, O. G., and Ovchinnikov, Y. A. 1983. Rapid synthesis of long-chain deoxyribooligonucleotides by the N-methylimidazole phosphotriester method. Nucl. Acids Res. 11:8369-8387.), NPE carbonate linker arm cleavable with DBU/pyridine (Eritja, R., Robles, J., Fernandezforner, D., Albericio, F., Giralt, E., and Pedroso, E. 1991. NPE-resin, a new approach to the solid-phase synthesis of protected peptides and oligonucleotides. 1. Synthesis of the supports and their application to oligonucleotide synthesis. Tetrahedron Lett. 32:1511-1514.), 9-Fluorenylmethyl linker or phthaloyl linker arm cleavable with DBU (Avino, A., Garcia, R. G., Diaz, A., Albericio, F., and Eritja, R. 1996. A comparative study of supports for the synthesis of oligonucleotides without using ammonia. Nucleos. Nucleot. 15:1871-1889; Brown, T., Pritchard, C. E., Turner, G., and Salisbury, S. A. 1989. A new base-stable linker for solid- phase oligonucleotide synthesis. J. Chem. Soc. Chem. Commun. 891-893.), Oxalyl linker, cleavable under very mild conditions (Alul, R. H., Singman, C. N., Zhang, G. R., and Letsinger, R. L. 1991. Oxalyl-CPG—A labile support for synthesis of sensitive oligonucleotide derivatives. Nucl. Acids Res. 19:1527-1532.), Malonic acid linker for the synthesis of 3′-phosphorylated oligonucleotides (Guzaev, A. and Lonnberg, H. 1997. A novel solid support for synthesis of 3′-phosphorylated chimeric oligonucleotides containing internucleosidic methyl phosphotriester and methyl-phosphonate linkages. Tetrahedron Lett. 38:3989-3992.), Diglycolic acid linker used to make 3′-TAMRA dye-labeled oligonucleotides (Mullah, B., Livak, K., Andrus, A., and Kenney, P. 1998. Efficient synthesis of double dye-labeled oligodeoxyribonucleotide probes and their application in a real time PCR assay. Nucl. Acids Res. 26:1026-1031.), Hydroquinone-O,O′-diacetic acid (Q-linker), which can be used for routine oligonucleotides to improve synthesis productivity or to synthesize base-labile products (Pon, R. T. and Yu, S. 1997a. Hydroquinone-O,O′-diacetic acid (‘Q-linker’) as a replacement for succinyl and oxalyl linker arms in solid phase oligonucleotide synthesis. Nucl. Acids Res. 25:3629-3635.).

In some instances, spacer comprises a polyethylene glycol polymer (PEG) having a molecular weight range of about 300 daltons, about 400 daltons, about 500 daltons, about 600 daltons, about 700 daltons, about 800 daltons, about 900 daltons, about 1,000 daltons to 8,000 daltons. The PEG may have a molecular weight of about 300 daltons, about 500 daltons, about 800 daltons, about 1000 daltons, about 1500 daltons, about 2000 daltons, about 3500 daltons, about 5000 daltons, or about 8000 daltons. In some embodiments, the PEG can range from about 5 repeats to about 150 repeats, about 8 repeats up to about 125 repeats.

In some embodiments, the spacer comprises a photolabile linker, a fluoride ion labile linker, or a cleavable linker. In embodiments, the spacer may comprise a benzenesulfonylethyl linker, an o-nitrobenzyl carbonate photolabile linker, a 5-methoxy-2-nitrobenzyl carbonate photolabile linker, an o-nitrophenyl-1,3-propanediol base photolabile linker, a fluoride ion labile diisopropylsilyl linker, a fluoride ion labile disiloxyl phosphoramidite linker, a NPE carbonate linker, a 9-fluorenylmethyl linker, a phthaloyl linker, an oxalyl linker, a malonic acid linker, a succinyl linker, a diglycolic acid linker, a hydroquinone-O,O′-diacetic acid (Q-linker), or a thiophospate linker. In particular embodiments, the spacer comprises a benzenesulfonylethyl linker cleavable with triethylamine/dioxane, or a spacer comprises a nonyl phenol ethoxylate (NPE) carbonate linker cleavable with 1,8-Diazabicyclo[5.4.0]undec-7-ene (DBU)/pyridine. In particular embodiments, the spacer comprises a 9-fluorenylmethyl linker or a phthaloyl linker cleavable with DBU. In some embodiments, the spacer comprises a succinic acid linked to an N-methylglycine (sarcosine) derivatized support, a succinic acid linked to 1,6-bis methylaminohexane spacer, a succinic acid linked to N-propyl polyethylene glycol Tentagel support, or a succinyl-sarcosine linkage.

The spacer may be grafted onto the solid support via an amine linkage, a secondary amine linkage, a thioether linkage, an ether linkage, a carbamate linkage, or an amide linkage, using the methods as disclosed herein.

### Agents/Labels

The solid support further comprises an agent. The agent may be attached to a solid support using a cleavable linker. In preferred embodiments, the solid support may be functionalized to permit covalent attachment of the agent and/or label. In some instances, a label (or multiple copies of the same label) and the agent are attached to the same solid support. Labels and/or agents may be attached to each other or to solid supports using cleavable linkers.

An agent can be any moiety or entity that can be associated with, including attached to, a unique label. An agent may be a single entity or it may be a plurality of entities. An agent may be a nucleic acid, a peptide, a protein, a cell, a cell lysate, a solid support, a polymer, a chemical, and the like, or an agent may be a plurality of any of the foregoing, or it may be a mixture of any of the foregoing. As an example, an agent may be nucleic acids (e.g., mRNA transcripts and/or genomic DNA fragments), solid supports such as beads or polymers, and/or proteins from a single cell or from a single cell population (e.g., a tumor or non-tumor tissue sample). An agent may also comprise a spacer as described herein.

In some embodiments, an agent is a nucleic acid. The nucleic acid agent may be single-stranded (ss) or double-stranded (ds), or it may be partially single-stranded and partially double-stranded. Nucleic acid agents include but are not limited to DNA such as genomic DNA fragments, PCR and other amplification products, RNA, cDNA, and the like. Nucleic acid agents may be fragments of larger nucleic acids such as but not limited to genomic DNA fragments. Accordingly, a portion or fragment when used in reference to a nucleotide sequence typically refers to smaller subsets of that nucleotide sequence. For example, such portions or fragments may range in size from 5 nucleotide residues to the entire nucleotide sequence minus one nucleic acid residue.

Nucleic acid sequence, nucleotide sequence, and nucleic acid molecule as used herein may interchangeably refer to an oligonucleotide or polynucleotide, and fragments or portions thereof, and to DNA or RNA of genomic or synthetic origin which may be single- or double-stranded, and represent the sense or antisense strand.

An isolated nucleic acid can refer to any nucleic acid molecule that has been removed from its natural state (e.g., removed from a cell and is, in a preferred embodiment, free of other genomic nucleic acid).

In some particular embodiments, the agent is a surface reactive nucleic acid molecule. The one or more surface reactive nucleic acid molecules may comprise an ISPCR Primer, a Barcode, a Unique Molecular Identifier (UMI) and/or a Universal Sequence. The surface reactive nucleic acid molecule may include oligonucleotides, nucleotides, analogs thereof, a molecular barcode, a Unique Molecular Identifier, a oligodT, an amplification primer, a cell type specific sequence, a pathogen-specific sequence, or a TCR specific sequence.

In some aspects, the invention provides a solid support, a population of solid support, or a composition comprising a solid support or a composition comprising a population of solid support having a surface reactive nucleic acid molecule, wherein the surface reactive nucleic acid molecule(s) comprise sequences of an ISPCR primer, a barcode, a unique molecular identifier (UMI), and a universal sequence. In some aspects, the surface reactive nucleic acid molecule(s) comprises one or more of the following: oligonucleotides, nucleotides, analogs thereof; a molecular barcode; a unique molecular identifier; a oligodT; an amplification primer; a cell type specific sequence; a primer for a specific gene, anywhere within the gene, or near a feature of interest, for example a mutation or translocation, primer for a pool of genes or features, a pathogen-specific sequence; a BCR specific sequence or a TCR specific sequence. In particular embodiments, the cell type specific sequence comprises the sequences adjacent to a feature of interest, mutation specific sequences, gene pools (gene synthesis libararies). In some aspects, the molecular barcode comprises approximately 12, 15, or 21 base pairs. In some aspects, the UMI comprises approximately 8 base pairs. In another aspect, the oligodT comprises approximately 30 base pairs. In particular embodiments, stoichiometrically mixed primers analogous to sequences of interest of interest are utilized when making the solid supports.

### Barcodes and Unique Molecular Identifiers

A barcode as used herein refers to a short sequence of nucleotides (for example, DNA or RNA) that is used as an identifier for an associated molecule, such as a target molecule and/or target nucleic acid, or as an identifier of the source of an associated molecule, such as a cell-of-origin. A barcode may also refer to any unique, non-naturally occurring, nucleic acid sequence that may be used to identify the originating source of a nucleic acid fragment. Although it is not necessary to understand the mechanism of an invention, it is believed that the barcode sequence provides a high-quality individual read of a barcode associated with a single cell, a viral vector, labeling ligand (e.g., an aptamer), protein, shRNA, sgRNA or cDNA such that multiple species can be sequenced together.

In preferred embodiments, sequencing is performed using unique molecular identifiers (UMI) which may be associated with the solid supports disclosed herein. The term “unique molecular identifiers” (UMI) as used herein refers to a sequencing linker or a subtype of nucleic acid barcode used in a method that uses molecular tags to detect and quantify unique amplified products. A UMI is used to distinguish effects through a single clone from multiple clones. A clone may refer to a single mRNA or target nucleic acid to be sequenced. The UMI may also be used to determine the number of transcripts that gave rise to an amplified product, or in the case of target barcodes as described herein, the number of binding events. In preferred embodiments, the amplification is by PCR or multiple displacement amplification (MDA).

In certain embodiments, an UMI with a random sequence of between 4 and 20 base pairs is added to a template, which is amplified and sequenced. In preferred embodiments, the UMI is added to the 5′ end of the template. Sequencing allows for high resolution reads, enabling accurate detection of true variants. As used herein, a true variant will be present in every amplified product originating from the original clone as identified by aligning all products with a UMI. Each clone amplified will have a different random UMI that will indicate that the amplified product originated from that clone. Not being bound by a theory, the UMI's are designed such that assignment to the original can take place despite up to 4-7 errors during amplification or sequencing. Not being bound by a theory, an UMI may be used to discriminate between true barcode sequences.

Unique molecular identifiers can be used, for example, to normalize samples for variable amplification efficiency. For example, in various embodiments, featuring a solid or semisolid support (for example a hydrogel bead), to which nucleic acid barcodes (for example a plurality of barcodes sharing the same sequence) are attached, each of the barcodes may be further coupled to a unique molecular identifier, such that every barcode on the particular solid or semisolid support receives a distinct unique molecule identifier. A unique molecular identifier can then be, for example, transferred to a target molecule with the associated barcode, such that the target molecule receives not only a nucleic acid barcode, but also an identifier unique among the identifiers originating from that solid or semisolid support.

A nucleic acid barcode or UMI can have a length of at least, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 nucleotides, and can be in single- or double-stranded form. Target molecule and/or target nucleic acids can be labeled with multiple nucleic acid barcodes in combinatorial fashion, such as a nucleic acid barcode concatemer. Typically, a nucleic acid barcode is used to identify a target molecule and/or target nucleic acid as being from a particular discrete volume, having a particular physical property (for example, affinity, length, sequence, etc.), or having been subject to certain treatment conditions. Target molecule and/or target nucleic acid can be associated with multiple nucleic acid barcodes to provide information about all of these features (and more). Each member of a given population of UMIs, on the other hand, is typically associated with (for example, covalently bound to or a component of the same molecule as) individual members of a particular set of identical, specific (for example, discrete volume-, physical property-, or treatment condition-specific) nucleic acid barcodes. Thus, for example, each member of a set of origin-specific nucleic acid barcodes, or other nucleic acid identifier or connector oligonucleotide, having identical or matched barcode sequences, may be associated with (for example, covalently bound to or a component of the same molecule as) a distinct or different UMI.

As disclosed herein, unique nucleic acid identifiers are used to label the target molecules and/or target nucleic acids, for example origin-specific barcodes and the like. The nucleic acid identifiers, nucleic acid barcodes, can include a short sequence of nucleotides that can be used as an identifier for an associated molecule, location, or condition. In certain embodiments, the nucleic acid identifier further includes one or more unique molecular identifiers and/or barcode receiving adapters. A nucleic acid identifier can have a length of about, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 base pairs (bp) or nucleotides (nt). In certain embodiments, a nucleic acid identifier can be constructed in combinatorial fashion by combining randomly selected indices (for example, about 1, 2, 3, 4, 5, 6, 7, 8, 9, or 10 indexes). Each such index is a short sequence of nucleotides (for example, DNA, RNA, or a combination thereof) having a distinct sequence. An index can have a length of about, for example, 4, 5, 6, 7, 8, 9, 10, 11, 12, 13, 14, 15, 16, 17, 18, 19, 20, 21, 22, 23, 24, or 25 bp or nt. Nucleic acid identifiers can be generated, for example, by split-pool synthesis methods, such as those described, for example, in International Patent Publication Nos. WO 2014/047556 and WO 2014/143158, each of which is incorporated by reference herein in its entirety.

One or more nucleic acid identifiers (for example a nucleic acid barcode) can be attached, or “tagged,” to a target molecule. This attachment can be direct (for example, covalent or noncovalent binding of the nucleic acid identifier to the target molecule) or indirect (for example, via an additional molecule). Such indirect attachments may, for example, include a barcode bound to a specific-binding agent that recognizes a target molecule. In certain embodiments, a barcode is attached to protein G and the target molecule is an antibody or antibody fragment. Attachment can be performed according to methods described herein.

Target molecules can be optionally labeled with multiple barcodes in combinatorial fashion (for example, using multiple barcodes bound to one or more specific binding agents that specifically recognizing the target molecule), thus greatly expanding the number of unique identifiers possible within a particular barcode pool.

In some embodiments, a nucleic acid identifier (for example, a nucleic acid barcode) may be attached to sequences that allow for amplification and sequencing (for example, SBS3 and P5 elements for Illumina sequencing). In certain embodiments, a nucleic acid barcode can further include a hybridization site for a primer (for example, a single-stranded DNA primer) attached to the end of the barcode. For example, an origin-specific barcode may be a nucleic acid including a barcode and a hybridization site for a specific primer. In particular embodiments, a set of origin-specific barcodes includes a unique primer specific barcode made, for example, using a randomized oligo type

A nucleic acid identifier can further include a unique molecular identifier and/or additional barcodes specific to, for example, a common support to which one or more of the nucleic acid identifiers are attached. Thus, a pool of target molecules can be added, for example, to a discrete volume containing multiple solid or semisolid supports (for example, beads) representing distinct treatment conditions (and/or, for example, one or more additional solid or semisolid support can be added to the discreet volume sequentially after introduction of the target molecule pool), such that the precise combination of conditions to which a given target molecule was exposed can be subsequently determined by sequencing the unique molecular identifiers associated with it.

Labeled target molecules and/or target nucleic acids associated origin-specific nucleic acid barcodes (optionally in combination with other nucleic acid barcodes as described herein) can be amplified by methods known in the art, such as polymerase chain reaction (PCR). For example, the nucleic acid barcode can contain universal primer recognition sequences that can be bound by a PCR primer for PCR amplification and subsequent high-throughput sequencing. In certain embodiments, the nucleic acid barcode includes or is linked to sequencing adapters (for example, universal primer recognition sequences) such that the barcode and sequencing adapter elements are both coupled to the target molecule. In particular examples, the sequence of the origin specific barcode is amplified, for example using PCR. In some embodiments, an origin-specific barcode further comprises a sequencing adaptor. In some embodiments, an origin-specific barcode further comprises universal priming sites.

### Barcodes Reversibly Coupled to Solid Substrate

In some embodiments, the origin-specific barcodes are reversibly coupled to a solid or semisolid substrate. In some embodiments, the origin-specific barcodes further comprise a nucleic acid capture sequence that specifically binds to the target nucleic acids and/or a specific binding agent that specifically binds to the target molecules. In specific embodiments, the origin-specific barcodes include two or more populations of origin-specific barcodes, wherein a first population comprises the nucleic acid capture sequence and a second population comprises the specific binding agent that specifically binds to the target molecules. In some examples, the first population of origin-specific barcodes further comprises a target nucleic acid barcode, wherein the target nucleic acid barcode identifies the population as one that labels nucleic acids. In some examples, the second population of origin-specific barcodes further comprises a target molecule barcode, wherein the target molecule barcode identifies the population as one that labels target molecules.

Barcode with Cleavage Sites

A nucleic acid barcode may be cleavable from a specific binding agent, for example, after the specific binding agent has bound to a target molecule. In some embodiments, the origin-specific barcode further comprises one or more cleavage sites. In some examples, at least one cleavage site is oriented such that cleavage at that site releases the origin-specific barcode from a substrate, such as a bead, for example a hydrogel bead, to which it is coupled. In some examples, at least one cleavage site is oriented such that the cleavage at the site releases the origin-specific barcode from the target molecule specific binding agent. In some examples, a cleavage site is an enzymatic cleavage site, such an endonuclease site present in a specific nucleic acid sequence. In other embodiments, a cleavage site is a peptide cleavage site, such that a particular enzyme can cleave the amino acid sequence. In still other embodiments, a cleavage site is a site of chemical cleavage.

### Barcode Adapters

In some embodiments, the target molecule is attached to an origin-specific barcode receiving adapter, such as a nucleic acid. In some examples, the origin-specific barcode receiving adapter comprises an overhang and the origin-specific barcode comprises a sequence capable of hybridizing to the overhang. A barcode receiving adapter is a molecule configured to accept or receive a nucleic acid barcode, such as an origin-specific nucleic acid barcode. For example, a barcode receiving adapter can include a single-stranded nucleic acid sequence (for example, an overhang) capable of hybridizing to a given barcode (for example, an origin-specific barcode), for example, via a sequence complementary to a portion or the entirety of the nucleic acid barcode. In certain embodiments, this portion of the barcode is a standard sequence held constant between individual barcodes. The hybridization couples the barcode receiving adapter to the barcode. In some embodiments, the barcode receiving adapter may be associated with (for example, attached to) a target molecule. As such, the barcode receiving adapter may serve as the means through which an origin-specific barcode is attached to a target molecule. A barcode receiving adapter can be attached to a target molecule according to methods known in the art. For example, a barcode receiving adapter can be attached to a polypeptide target molecule at a cysteine residue (for example, a C-terminal cysteine residue). A barcode receiving adapter can be used to identify a particular condition related to one or more target molecules, such as a cell of origin or a discreet volume of origin. For example, a target molecule can be a cell surface protein expressed by a cell, which receives a cell-specific barcode receiving adapter. The barcode receiving adapter can be conjugated to one or more barcodes as the cell is exposed to one or more conditions, such that the original cell of origin or well origin for the target molecule, as well as each condition to which the cell or well was exposed, can be subsequently determined by identifying the sequence of the barcode receiving adapter/barcode concatemer.

Barcode with Capture Moiety

In some embodiments, an origin-specific barcode further includes a capture moiety, covalently or non-covalently linked. In specific embodiments, a targeting probe is labeled with biotin, for instance by incorporation of biotin-16-UTP during in vitro transcription, allowing later capture by streptavidin. Other means for labeling, capturing, and detecting an origin-specific barcode include: incorporation of aminoallyl-labeled nucleotides, incorporation of sulfhydryl-labeled nucleotides, incorporation of allyl- or azide-containing nucleotides.

### Other Barcoding Embodiments

DNA barcoding is based on a relatively simple concept. For example, most eukaryote cells contain mitochondria, and mitochondrial DNA (mtDNA) has a relatively fast mutation rate, which results in significant variation in mtDNA sequences between species and, in principle, a comparatively small variance within species. A 648-bp region of the mitochondrial cytochrome c oxidase subunit 1 (CO1) gene was proposed as a potential ‘barcode’. As of 2009, databases of CO1 sequences included at least 620,000 specimens from over 58,000 species of animals, larger than databases available for any other gene. Ausubel, J., “A botanical macroscope” Proceedings of the National Academy of Sciences 106(31):12569 (2009).

Additionally, other barcoding designs and tools have been described (see e.g., Birrell et al., (2001) Proc. Natl Acad. Sci. USA 98, 12608-12613; Giaever, et al., (2002) Nature 418, 387-391; Winzeler et al., (1999) Science 285, 901-906; and Xu et al., (2009) Proc Natl Acad Sci USA. February 17; 106(7):2289-94).

In some embodiments, the agent is associated with a unique label of the functionalized solid support or the population of functionalized solid support. Associated can refer to a relationship between the agent and the unique label such that the unique label may be used to identify the agent, identify the source or origin of the agent, identify one or more conditions to which the agent has been exposed, etc. A label that is associated with an agent may be, for example, physically attached to the agent, either directly or indirectly, or it may be in the same defined, typically physically separate, volume as the agent. A defined volume may be an emulsion droplet, a well (for example of a multiwell plate), a tube, a container, and the like. It is to be understood that the defined volume will typically contain only one agent and the label with which it is associated, although a volume containing multiple agents with multiple copies of the label is also contemplated depending on the application.

An agent may be associated with a single copy of a unique label or it may be associated with multiple copies of the same unique label including for example 2, 3, 4, 5, 6, 7, 8, 9, 10, 100, 1000, 10,000, 100,000 or more copies of the same unique label. In this context, the label is considered unique because it is different from labels associated with other, different agents.

Attachment of labels to agents may be direct or indirect. The attachment chemistry will depend on the nature of the agent and/or any derivatization or functionalization applied to the agent. For example, labels can be directly attached through covalent attachment. The label may include a moiety, which may be a non-nucleotide chemical modification, to facilitate attachment. By way of non-limiting example, the label may include methylated nucleotides, uracil bases, phosphorothioate groups, ribonucleotides, diol linkages, disulfide linkages, etc., to enable covalent attachment to an agent.

The terms coupled, connected, attached, linked, or conjugated are used interchangeably herein and encompass direct as well as indirect connection, attachment, linkage, or conjugation unless the context clearly dictates otherwise. The attachment of a ligand to a bead may be covalent or non-covalent. Attachment may be reversible or irreversible. Such attachment includes, but is not limited to, covalent bonding, ionic bonding, Van der Waals forces or friction, and the like.

Methods for attaching nucleic acids to each other, as for example attaching nucleic acid labels to nucleic acid agents, are known in the art. Such methods include but are not limited to ligation, such as blunt end ligation or cohesive overhang ligation, and polymerase-mediated attachment methods (see, e.g., U.S. Pat. Nos. 7,863,058 and 7,754,429; Green and Sambrook. Molecular Cloning: A Laboratory Manual, Fourth Edition, 2012; Current Protocols in Molecular Biology, and Current Protocols in Nucleic Acid Chemistry, all of which are incorporated herein by reference).

### Adapter

In some embodiments, oligonucleotide adapters are used to attach a unique label to an agent or to a solid support. In some embodiments, an oligonucleotide adapter comprises one or more known sequences, e.g., an amplification sequence, a capture sequence, a primer sequence, and the like. In some embodiments, the adapter comprises a thymidine (T) tail overhang. Methods for producing a thymidine tail overhang are known in the art, e.g., using terminal deoxynucleotide transferase (TdT) or a polymerase that adds a thymidine overhang at the termination of polymerization. In some embodiments, the oligonucleotide adapter comprises a region that is forked.

In some embodiments, the adapter comprises a capture or detection moiety. Examples of such moieties include, but are not limited to, fluorophores, microparticles such as quantum dots (Empodocles, et al., Nature 399:126-130, 1999), gold nanoparticles (Reichert et al., Anal. Chem. 72:6025-6029, 2000), microbeads (Lacoste et al., Proc. Natl. Acad. Sci. USA 97(17):9461-9466, 2000), biotin, DNP (dinitrophenyl), fucose, digoxigenin, and other moieties known to those skilled in the art. In some embodiments, the moiety is biotin.

### Detectable Oligonucleotide Tags

The unique labels of the invention are, at least in part, nucleic acid in nature, and are generated by sequentially attaching two or more detectable oligonucleotide tags to each other. As used herein, a detectable oligonucleotide tag is an oligonucleotide that can be detected by sequencing of its nucleotide sequence and/or by detecting non-nucleic acid detectable moieties it may be attached to.

The oligonucleotide tags are typically randomly selected from a diverse plurality of oligonucleotide tags. In some instances, an oligonucleotide tag may be present once in a plurality or it may be present multiple times in a plurality. In the latter instance, the plurality of tags may be comprised of a number of subsets each comprising a plurality of identical tags. In some important embodiments, these subsets are physically separate from each other. Physical separation may be achieved by providing the subsets in separate wells of a multiwell plate or separate droplets from an emulsion. It is the random selection and thus combination of oligonucleotide tags that results in a unique label. Accordingly, the number of distinct (i.e., different) oligonucleotide tags required to uniquely label a plurality of agents can be far less than the number of agents being labeled. This is particularly advantageous when the number of agents is large (e.g., when the agents are members of a library).

The oligonucleotide tags may be detectable by virtue of their nucleotide sequence, or by virtue of a non-nucleic acid detectable moiety that is attached to the oligonucleotide such as but not limited to a fluorophore, or by virtue of a combination of their nucleotide sequence and the non-nucleic acid detectable moiety.

An oligonucleotide may be a nucleic acid such as deoxyribonucleic acid (DNA), ribonucleic acid (RNA), or DNA/RNA hybrids and includes analogs of either DNA or RNA made from nucleotide analogs known in the art (see, e.g. U.S. Patent or Patent Application Publications: U.S. Pat. Nos. 7,399,845, 7,741,457, 8,022,193, 7,569,686, 7,335,765, 7,314,923, 7,335,765, and 7,8163,33, US 20110009471, the entire contents of each of which are incorporated herein by reference). Oligonucleotides may be single-stranded (such as sense or antisense oligonucleotides), double-stranded, or partially single-stranded and partially double-stranded.

In some embodiments, a detectable oligonucleotide tag comprises one or more non-oligonucleotide detectable moieties. Examples of detectable moieties include, but are not limited to, fluorophores, microparticles including quantum dots (Empodocles, et al., Nature 399:126-130, 1999), gold nanoparticles (Reichert et al., Anal. Chem. 72:6025-6029, 2000), microbeads (Lacoste et al., Proc. Natl. Acad. Sci. USA 97(17):9461-9466, 2000), biotin, DNP (dinitrophenyl), fucose, digoxigenin, haptens, and other detectable moieties known to those skilled in the art. In some embodiments, the detectable moieties are quantum dots. Methods for detecting such moieties are described herein and/or are known in the art.

Thus, detectable oligonucleotide tags may be, but are not limited to, oligonucleotides comprising unique nucleotide sequences, oligonucleotides comprising detectable moieties, and oligonucleotides comprising both unique nucleotide sequences and detectable moieties.

A unique nucleotide sequence may be a nucleotide sequence that is different (and thus distinguishable) from the sequence of each detectable oligonucleotide tag in a plurality of detectable oligonucleotide tags. A unique nucleotide sequence may also be a nucleotide sequence that is different (and thus distinguishable) from the sequence of each detectable oligonucleotide tag in a first plurality of detectable oligonucleotide tags but identical to the sequence of at least one detectable oligonucleotide tag in a second plurality of detectable oligonucleotide tags. A unique sequence may differ from other sequences by multiple bases (or base pairs). The multiple bases may be contiguous or non-contiguous. Methods for obtaining nucleotide sequences (e.g., sequencing methods) are described herein and/or are known in the art.

In embodiments, detectable oligonucleotide tags comprise one or more of a ligation sequence, a priming sequence, a capture sequence, and a unique sequence. A ligation sequence is a sequence complementary to a second nucleotide sequence which allows for ligation of the detectable oligonucleotide tag to another entity comprising the second nucleotide sequence, e.g., another detectable oligonucleotide tag or an oligonucleotide adapter. A priming sequence is a sequence complementary to a primer, e.g., an oligonucleotide primer used for an amplification reaction such as but not limited to PCR. A capture sequence is a sequence capable of being bound by a capture entity. A capture entity may be an oligonucleotide comprising a nucleotide sequence complementary to a capture sequence, e.g. a second detectable oligonucleotide tag or an oligonucleotide attached to a bead. A capture entity may also be any other entity capable of binding to the capture sequence, e.g. an antibody or peptide. An index sequence is a sequence comprising a unique nucleotide sequence and/or a detectable moiety as described above. A capture entity can therefore be any molecule capable of attaching and/or binding to a nucleic acid (i.e., for example, a barcode nucleic acid). For example, a capture probe may be an oligonucleotide attached to a bead, wherein the oligonucleotide is at least partially complementary to another oligonucleotide. Alternatively, a capture probe may comprise a polyethylene glycol linker, an antibody, a polyclonal antibody, a monoclonal antibody, a Fab fragment, a biological receptor complex, an enzyme, a hormone, an antigen, and/or a fragment or portion thereof.

The nucleic acids may be bound to the support by hybridizing the capture sequence to a complementary sequence covalently attached to the support. The capture sequence (also referred to as a universal capture sequence) is a nucleic acid sequence complementary to a sequence attached to a support that may dually serve as a universal primer. In some aspects, the universal primer sequence is synthesized at the 3′ end of the oligonucleotide sequences bound to the solid support to enable stoichiometric addition of diverse oligonucleotide capture sequences to mRNA capture beads. In an aspect, photocleavable linkers may be incorporated within the oligonucleotide sequences bound to the solid support.

A label or detectable label can refer to any composition detectable by spectroscopic, photochemical, biochemical, immunochemical, electrical, optical or chemical means. Such labels include biotin for staining with labeled streptavidin conjugate, magnetic beads (e.g., Dynabeads®), fluorescent dyes (e.g., fluorescein, texas red, rhodamine, green fluorescent protein, and the like), radiolabels (e.g., 3H, 1251, 35S, 14C, or 32P), enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. Patents teaching the use of such labels include, but are not limited to, U.S. Pat. Nos. 3,817,837; 3,850,752; 3,939,350; 3,996,345; 4,277,437; 4,275,149; and 4,366,241 (all herein incorporated by reference). The labels contemplated in the present invention may be detected by many methods. For example, radiolabels may be detected using photographic film or scintillation counters, fluorescent markers may be detected using a photodetector to detect emitted light. Enzymatic labels are typically detected by providing the enzyme with a substrate and detecting, the reaction product produced by the action of the enzyme on the substrate, and calorimetric labels are detected by simply visualizing the colored label. Examples of the labeling substance which may be employed include labeling substances known to those skilled in the art, such as fluorescent dyes, enzymes, coenzymes, chemiluminescent substances, and radioactive substances. Specific examples include radioisotopes (e.g., 32P, 14C, 125I, 3H, and 131I), fluorescein, rhodamine, dansyl chloride, umbelliferone, luciferase, peroxidase, alkaline phosphatase, β-galactosidase, β-glucosidase, horseradish peroxidase, glucoamylase, lysozyme, saccharide oxidase, microperoxidase, biotin, and ruthenium. In the case where biotin is employed as a labeling substance, preferably, after addition of a biotin-labeled antibody, streptavidin bound to an enzyme (e.g., peroxidase) is further added. Advantageously, the label is a fluorescent label. Examples of fluorescent labels include, but are not limited to, Atto dyes, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinyl sulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3 -(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5 -[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′ tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine. A fluorescent label may be a fluorescent protein, such as blue fluorescent protein, cyan fluorescent protein, green fluorescent protein, red fluorescent protein, yellow fluorescent protein or any photoconvertible protein. Colorimetric labeling, bioluminescent labeling and/or chemiluminescent labeling may further accomplish labeling. Labeling further may include energy transfer between molecules in the hybridization complex by perturbation analysis, quenching, or electron transport between donor and acceptor molecules, the latter of which may be facilitated by double stranded match hybridization complexes. The fluorescent label may be a perylene or a terrylen. In the alternative, the fluorescent label may be a fluorescent bar code. Advantageously, the label may be light sensitive, wherein the label is light-activated and/or light cleaves the one or more linkers to release the molecular cargo. The light-activated molecular cargo may be a major light-harvesting complex (LHCII). In another embodiment, the fluorescent label may induce free radical formation.

Advantageously, agents may be uniquely labeled in a dynamic manner (see, e.g., U.S. Provisional Patent Application No. 61/703,884 filed Sep. 21, 2012). The unique labels are, at least in part, nucleic acid in nature, and may be generated by sequentially attaching two or more detectable oligonucleotide tags to each other and each unique label may be associated with a separate agent. A detectable oligonucleotide tag may be an oligonucleotide that may be detected by sequencing of its nucleotide sequence and/or by detecting non-nucleic acid detectable moieties to which it may be attached. Oligonucleotide tags may be detectable by virtue of their nucleotide sequence, or by virtue of a non-nucleic acid detectable moiety that is attached to the oligonucleotide such as but not limited to a fluorophore, or by virtue of a combination of their nucleotide sequence and the non-nucleic acid detectable moiety. A detectable oligonucleotide tag may comprise one or more non-oligonucleotide detectable moieties. Examples of detectable moieties may include, but are not limited to, fluorophores, microparticles including quantum dots (Empodocles, et al., Nature 399:126-130, 1999), gold nanoparticles (Reichert et al., Anal. Chem. 72:6025-6029, 2000), microbeads (Lacoste et al., Proc. Natl. Acad. Sci. USA 97(17):9461-9466, 2000), biotin, DNP (dinitrophenyl), fucose, digoxigenin, haptens, and other detectable moieties known to those skilled in the art.

In some embodiments, the detectable moieties may be quantum dots. Methods for detecting such moieties are described herein and/or are known in the art. Thus, detectable oligonucleotide tags may be, but are not limited to, oligonucleotides which may comprise unique nucleotide sequences, oligonucleotides which may comprise detectable moieties, and oligonucleotides which may comprise both unique nucleotide sequences and detectable moieties. A unique label may be produced by sequentially attaching two or more detectable oligonucleotide tags to each other. The detectable tags may be present or provided in a plurality of detectable tags. The same or a different plurality of tags may be used as the source of each detectable tag may be part of a unique label. In other words, a plurality of tags may be subdivided into subsets and single subsets may be used as the source for each tag. One or more other species may be associated with the tags. In particular, nucleic acids released by a lysed cell may be ligated to one or more tags. These may include, for example, chromosomal DNA, RNA transcripts, tRNA, mRNA, mitochondrial DNA, or the like. Such nucleic acids may be sequenced or further processed according to methods disclosed herein, in addition to sequencing the tags themselves, which may yield information about the nucleic acid profile of the cells, which can be associated with the tags, or the conditions that the corresponding droplet or cell was exposed to.

### Methods for Functionalized Solid Support Synthesis

Methods of functionalizing the surface of a solid support are also provided and include reacting a solid support having surface bearing reacting groups with an activator, so as to obtain a solid support with an activated surface comprising an activating moiety, and reacting the activated surface with a spacer compound having a first moiety that reacts with the activating moiety and optionally a second moiety comprising a functional group whereby the reacting of this step b) obtains, on the solid support, a spacer grafted-thereon, whereby the surface of the solid support is functionalized. The method of functionalization of the surface can depend on the reacting groups on the surface of the solid support. In a preferred embodiment, the solid support surface bears hydroxyl reacting groups. Regardless of the reacting groups, activating the surface reacting groups should be performed in a manner to allow the functionalization of the solid support.

Following the step of activating of the surface of the solid support, the activated surface is reacted with a spacer compound comprising a first moiety that reacts with the activating moiety on the activated surface of the solid support. The spacer may also optionally contain a second moiety comprising a functional group that upon the reacting of the activated surface with the spacer compound having a first and second moiety, the solid support comprises a spacer grated-thereon, the solid support functionalized, with the second moiety comprising a functional group that can be exposed for further reaction.

### Activated Surface of the Solid Support

The surface of the solid support is activated and comprises a surface reactive nucleic acid molecule. In embodiments, the activated surface allows the functionalization and synthesis of capture beads. Activating the surface of the solid support can comprise activation of surface reacting groups allows functionalization and synthesis of capture beads. In one embodiment, the solid support may contain surface hydroxyls, e.g., primary and/or secondary hydroxyls. The surface hydroxyls of the solid support can be activated using common reagents, for example, CNBr, Carbonyl Diimidazole, tresyl chloride, and divinylsulfone, and as described, for example at [0149]-[0157] of International Patent Publication No. WO 2019084058, incorporated by reference in its entirety. Synthesis of the label may occur while an agent is being exposed to one or more conditions such that one or more steps of the syntheses disclosed herein may be performed in different order, or simultaneously. In an embodiment of the invention, the bead synthesized by the method described herein has a higher capture efficiency compared to the capture efficiency of a commercially available bead.

### Oligonucleotide Syntheses

In some preferred embodiments, an oligonucleotide is preferably attached as one or more agents on the solid support. Oligonucleotide synthesis can be performed by one of the exemplary methods discussed herein, and may be synthesized in the 5′ to 3′ direction or the 3′ to 5′ direction. In an aspect, the solid support is provided with an oligonucleotide handle onto which additional oligosynthesis is performed.

Amplification can be any suitable production of additional copies of a nucleic acid sequence and is generally carried out using polymerase chain reaction. Dieffenbach C. W. and G. S. Dveksler (1995) In: PCR Primer, a Laboratory Manual, Cold Spring Harbor Press, Plainview, N.Y.

Polymerase chain reaction (“PCR”) refers to the method of K. B. Mullis U.S. Pat. Nos. 4,683,195 and 4,683,202, herein incorporated by reference, which describe a method for increasing the concentration of a segment of a target sequence in a mixture of genomic DNA without cloning or purification. The length of the amplified segment of the desired target sequence is determined by the relative positions of two oligonucleotide primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”. With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of 32P-labeled deoxynucleotide triphosphates, such as dCTP or dATP, into the amplified segment). In addition to genomic DNA, any oligonucleotide sequence can be amplified with the appropriate set of primer molecules. In particular, the amplified segments created by the PCR process itself are, themselves, efficient templates for subsequent PCR amplifications.

A probe typically refers to an oligonucleotide (i.e., a sequence of nucleotides), whether occurring naturally as in a purified restriction digest or produced synthetically, recombinantly or by PCR amplification, which is capable of hybridizing to another oligonucleotide of interest. A probe may be single-stranded or double-stranded. Probes are useful in the detection, identification and isolation of particular gene sequences. It is contemplated that any probe used in the present invention will be labeled with any reporter molecule, so that it is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

Purified or isolated may refer to a component of a composition that has been subjected to treatment (i.e., for example, fractionation) to remove various other components. Where the term substantially purified is used, this designation will refer to a composition in which a nucleic acid sequence forms the major component of the composition, such as constituting about 50%, about 60%, about 70%, about 80%, about 90%, about 95% or more of the composition (i.e., for example, weight/weight and/or weight/volume). Purified to homogeneity is used to include compositions that have been purified to apparent homogeneity such that there is single nucleic acid species (i.e., for example, based upon SDS-PAGE or HPLC analysis). A purified composition is not intended to mean that some trace impurities may remain. Substantially purified refers to molecules, such as nucleic acid sequences, that are removed from their natural environment, isolated or separated, and are at least 60% free, preferably 75% free, and more preferably 90% free from other components with which they are naturally associated. An isolated polynucleotide is therefore a substantially purified polynucleotide.

DNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements which direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region. Advantageously, the methods disclosed herein allow for the production of solid supports comprising nucleic acid molecules in a 5′ to 3′ orientation or in a 3′ to 5′ orientation.

A poly A site or poly A sequence as used herein denotes a DNA sequence which directs both the termination and polyadenylation of the nascent RNA transcript. Efficient polyadenylation of the recombinant transcript is desirable as transcripts lacking a poly A tail are unstable and are rapidly degraded. The poly A signal utilized in an expression vector may be heterologous or endogenous. An endogenous poly A signal is one that is found naturally at the 3′ end of the coding region of a given gene in the genome. A heterologous poly A signal is one which is isolated from one gene and placed 3′ of another gene. Efficient expression of recombinant DNA sequences in eukaryotic cells involves expression of signals directing the efficient termination and polyadenylation of the resulting transcript. Transcription termination signals are generally found downstream of the polyadenylation signal and are a few hundred nucleotides in length.

Nucleic acid molecule encoding, DNA sequence encoding, and DNA encoding may interchangeably refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

In some preferred embodiments, activating is performed under dry conditions or non-aqueous conditions or solid phase synthesis conditions.

In some preferred embodiments, the UMI is added to the 5′ end of the template. When nucleic acid barcodes (for example a plurality of barcodes sharing the same sequence) are attached to the solid support, each of the barcodes may be further coupled to a unique molecular identifier, such that every barcode on the particular solid or semisolid support receives a distinct unique molecule identifier. A unique molecular identifier can then be, for example, transferred to a target molecule with the associated barcode, such that the target molecule receives not only a nucleic acid barcode, but also an identifier unique among the identifiers originating from that solid or semisolid support.

Target molecule and/or target nucleic acids can be labeled with multiple nucleic acid barcodes in combinatorial fashion, such as a nucleic acid barcode concatemer. Typically, a nucleic acid barcode is used to identify a target molecule and/or target nucleic acid as being from a particular discrete volume, having a particular physical property (for example, affinity, length, sequence, etc.), or having been subject to certain treatment conditions. Target molecule and/or target nucleic acid can be associated with multiple nucleic acid barcodes to provide information about all of these features (and more). Each member of a given population of UMIs, on the other hand, is typically associated with (for example, covalently bound to or a component of the same molecule as) individual members of a particular set of identical, specific (for example, discreet volume-, physical property-, or treatment condition-specific) nucleic acid barcodes. Thus, for example, each member of a set of origin-specific nucleic acid barcodes, or other nucleic acid identifier or connector oligonucleotide, having identical or matched barcode sequences, may be associated with (for example, covalently bound to or a component of the same molecule as) a distinct or different UMI.

As disclosed herein, unique nucleic acid identifiers are used to label the target molecules and/or target nucleic acids, for example origin-specific barcodes and the like. Nucleic acid identifiers can be generated, for example, by split-pool synthesis methods, such as those described, for example, in International Patent Publication Nos. WO 2014/047556 and WO 2014/143158, each of which is incorporated by reference herein in its entirety.

Reacting with Nucleotides

Reacting with dinucleotides or trinucleotides is envisioned and may allow for improved processing. Accordingly, it is envisioned as to or in the practice of the invention provides that there can be a method for preparing uniquely barcoded mRNA capture microbeads, which has a unique barcode and diameter suitable for microfluidic devices which may comprise: 1) performing reverse phosphoramidite synthesis on the surface of the bead in a pool-and-split fashion, such that in each cycle of synthesis the beads are split into four reactions with unique oligonucleotides, preferably of a length of two or more bases; 2) repeating this process a large number of times, in embodiments, at least four, at least six, or more to provide millions of unique barcodes on the surface of each bead in the pool. (See www.ncbi.nlm.nih.gov/pmc/articles/PMC206447). In particular embodiments, the process is repeated between about 1 and 150 times. Advantageously, using pairs or triplets of nucleotide bases reduces the amount of when beads not in inert atmosphere and transferred from vessels, likely contributing factor to bead loss and loss of quality (including increased truncations) and may only require 4 or 6 repeats of the process. Chemistry is also able to be performed more rapidly. In embodiments, an extension method is used with three premade barcodes, avoiding the split-and-pool methods, which can create potential losses, for example as described in Han et al., Cell, 172:5, p. 1091-1107 (2018), incorporated herein by reference.

One or more nucleic acid identifiers (for example a nucleic acid barcode) can be attached, or “tagged,” to a target molecule. This attachment can be direct (for example, covalent or noncovalent binding of the nucleic acid identifier to the target molecule) or indirect (for example, via an additional molecule). Such indirect attachments may, for example, include a barcode bound to a specific-binding agent that recognizes a target molecule. In certain embodiments, a barcode is attached to protein G and the target molecule is an antibody or antibody fragment. Attachment of a barcode to target molecules (for example, proteins and other biomolecules) can be performed using standard methods well known in the art. For example, barcodes can be linked via cysteine residues (for example, C-terminal cysteine residues). In other examples, barcodes can be chemically introduced into polypeptides (for example, antibodies) via a variety of functional groups on the polypeptide using appropriate group-specific reagents (see for example www.drmr.com/abcon). In certain embodiments, barcode tagging can occur via a barcode receiving adapter associate with (for example, attached to) a target molecule, as described herein.

Target molecules can be optionally labeled with multiple barcodes in combinatorial fashion (for example, using multiple barcodes bound to one or more specific binding agents that specifically recognizing the target molecule), thus greatly expanding the number of unique identifiers possible within a particular barcode pool. In certain embodiments, barcodes are added to a growing barcode concatemer attached to a target molecule, for example, one at a time. In other embodiments, multiple barcodes are assembled prior to attachment to a target molecule. Compositions and methods for concatemerization of multiple barcodes are described, for example, in International Patent Publication No. WO 2014/047561, which is incorporated herein by reference in its entirety.

In some embodiments, a nucleic acid identifier (for example, a nucleic acid barcode) may be attached to sequences that allow for amplification and sequencing (for example, SBS3 and P5 elements for Illumina sequencing). In certain embodiments, a nucleic acid barcode can further include a hybridization site for a primer (for example, a single-stranded DNA primer) attached to the end of the barcode. For example, an origin-specific barcode may be a nucleic acid including a barcode and a hybridization site for a specific primer. In particular embodiments, a set of origin-specific barcodes includes a unique primer specific barcode made, for example, using a randomized oligo type . Primers for specific genes can be utilized, which can include near a feature of interest, such as a mutation or translocation, or primers for pools of genes or features, for example a primer specific to a conserved region, a pool of genes. In certain embodiments, the primers can be designed for enrichment of particular pathways in a cell population, such as, for example in a disease, virus or bacterial infection. In embodiments, more than one capture sequence can be utilized for functionalization.

Primers for enrichment can be designed for microbial infections, including for example, bacterial infections. Primers can be designed for viral, bacterial and other infectious diseases, strains, or groups of strains. Other examples of interest can include enrichment using the functionalized solid supports with primers for immune checkpoints, gene expression in tumors, cancers- including loss of heterozygosity, and cancer drug resistance detection, and epigenetic modifications.

Examples of bacteria for which primers can be designed and utilized with the solid supports in the disclosed methods include without limitation any one or more of (or any combination of) Acinetobacter baumanii, Actinobacillus sp., Actinomycetes, Actinomyces sp. (such as Actinomyces israelii and Actinomyces naeslundii), Aeromonas sp. (such as Aeromonas hydrophila, Aeromonas veronii biovar sobria (Aeromonas sobria), and Aeromonas caviae), Anaplasma phagocytophilum, Anaplasma marginale Alcaligenes xylosoxidans, Acinetobacter baumanii, Actinobacillus actinomycetemcomitans, Bacillus sp. (such as Bacillus anthracis, Bacillus cereus, Bacillus subtilis, Bacillus thuringiensis, and Bacillus stearothermophilus), Bacteroides sp. (such as Bacteroides fragilis), Bartonella sp. (such as Bartonella bacilliformis and Bartonella henselae, Bifidobacterium sp., Bordetella sp. (such as Bordetella pertussis, Bordetella parapertussis, and Bordetella bronchi septica), Borrelia sp. (such as Borrelia recurrentis, and Borrelia burgdorferi), Brucella sp. (such as Brucella abortus, Brucella canis, Brucella melintensis and Brucella suis), Burkholderia sp. (such as Burkholderia pseudomallei and Burkholderia cepacia), Campylobacter sp. (such as Campylobacter jejuni, Campylobacter coli, Campylobacter lari and Campylobacter fetus), Capnocytophaga sp., Cardiobacterium hominis, Chlamydia trachomati s, Chlamydophila pneumoniae, Chlamydophila psittaci, Citrobacter sp. Coxiella burnetii, Corynebacterium sp. (such as, Corynebacterium diphtheriae, Corynebacterium jeikeum and Corynebacterium), Clostridium sp. (such as Clostridium perfringens, Clostridium difficile, Clostridium botulinum and Clostridium tetani), Eikenella corrodens, Enterobacter sp. (such as Enterobacter aerogenes, Enterobacter agglomerans, Enterobacter cloacae and Escherichia coli, including opportunistic Escherichia coli, such as enterotoxigenic E. coli, enteroinvasive E. coli, enteropathogenic E. coli, enterohemorrhagic E. coli, enteroaggregative E. coli and uropathogenic E. coli) Enterococcus sp. (such as Enterococcus faecalis and Enterococcus faecium) Ehrlichia sp. (such as Ehrlichia chafeensia and Ehrlichia canis), Epidermophyton floccosum, Erysipelothrix rhusiopathiae, Eubacterium sp., Francisella tularensis, Fusobacterium nucleatum, Gardnerella vaginalis, Gemella morbillorum, Haemophilus sp. (such as Haemophilus influenzae, Haemophilus ducreyi, Haemophilus aegyptius, Haemophilus parainfluenzae, Haemophilus haemolyticus and Haemophilus parahaemolyticus, Helicobacter sp. (such as Helicobacter pylori, Helicobacter cinaedi and Helicobacter fennelliae), Kingella kingii, Klebsiella sp. (such as Klebsiella pneumoniae, Klebsiella granulomatis and Klebsiella oxytoca), Lactobacillus sp., Listeria monocytogenes, Leptospira interrogans, Legionella pneumophila, Leptospira interrogans, Peptostreptococcus sp., Mannheimia hemolytica, Microsporum canis, Moraxella catarrhalis, Morganella sp., Mobiluncus sp., Micrococcus sp., Mycobacterium sp. (such as Mycobacterium leprae, Mycobacterium tuberculosis, Mycobacterium paratuberculosis, Mycobacterium intracellulare, Mycobacterium avium, Mycobacterium bovis, and Mycobacterium marinum), Mycoplasm sp. (such as Mycoplasma pneumoniae, Mycoplasma hominis, and Mycoplasma genitalium), Nocardia sp. (such as Nocardia asteroides, Nocardia cyriacigeorgica and Nocardia brasiliensis), Neisseria sp. (such as Neisseria gonorrhoeae and Neisseria meningitidis), Pasteurella multocida, Pityrosporum orbiculare (Malassezia furfur), Plesiomonas shigelloides. Prevotella sp., Porphyromonas sp., Prevotella melaninogenica, Proteus sp. (such as Proteus vulgaris and Proteus mirabilis), Providencia sp. (such as Providencia alcalifaciens, Providencia rettgeri and Providencia stuartii), Pseudomonas aeruginosa, Propionibacterium acnes, Rhodococcus equi, Rickettsia sp. (such as Rickettsia rickettsii, Rickettsia akari and Rickettsia prowazekii, Orientia tsutsugamushi (formerly: Rickettsia tsutsugamushi) and Rickettsia typhi), Rhodococcus sp., Serratia marcescens, Stenotrophomonas maltophilia, Salmonella sp. (such as Salmonella enterica, Salmonella typhi, Salmonella paratyphi, Salmonella enteritidis, Salmonella cholerasuis and Salmonella typhimurium), Serratia sp. (such as Serratia marcesans and Serratia liquifaciens), Shigella sp. (such as Shigella dysenteriae, Shigella flexneri, Shigella boydii and Shigella sonnei), Staphylococcus sp. (such as Staphylococcus aureus, Staphylococcus epidermidis, Staphylococcus hemolyticus, Staphylococcus saprophyticus), Streptococcus sp. (such as Streptococcus pneumoniae (for example chloramphenicol-resistant serotype 4 Streptococcus pneumoniae, spectinomycin-resistant serotype 6B Streptococcus pneumoniae, streptomycin-resistant serotype 9V Streptococcus pneumoniae, erythromycin-resistant serotype 14 Streptococcus pneumoniae, optochin-resistant serotype 14 Streptococcus pneumoniae, rifampicin-resistant serotype 18C Streptococcus pneumoniae, tetracycline-resistant serotype 19F Streptococcus pneumoniae, penicillin-resistant serotype 19F Streptococcus pneumoniae, and trimethoprim-resistant serotype 23F Streptococcus pneumoniae, chloramphenicol-resistant serotype 4 Streptococcus pneumoniae, spectinomycin-resistant serotype 6B Streptococcus pneumoniae, streptomycin-resistant serotype 9V Streptococcus pneumoniae, optochin-resistant serotype 14 Streptococcus pneumoniae, rifampicin-resistant serotype 18C Streptococcus pneumoniae, penicillin-resistant serotype 19F Streptococcus pneumoniae, or trimethoprim-resistant serotype 23F Streptococcus pneumoniae), Streptococcus agalactiae, Streptococcus mutans, Streptococcus pyogenes, Group A streptococci, Streptococcus pyogenes, Group B streptococci, Streptococcus agalactiae, Group C streptococci, Streptococcus anginosus, Streptococcus equismilis, Group D streptococci, Streptococcus bovis, Group F streptococci, and Streptococcus anginosus Group G streptococci), Spirillum minus, Streptobacillus moniliformi, Treponema sp. (such as Treponema carateum, Treponema petenue, Treponema pallidum and Treponema endemicum, Trichophyton rubrum, T. mentagrophytes, Tropheryma whippelii, Ureaplasma urealyticum, Veillonella sp., Vibrio sp. (such as Vibrio cholerae, Vibrio parahemolyticus, Vibrio vulnificus, Vibrio parahaemolyticus, Vibrio vulnificus, Vibrio alginolyticus, Vibrio mimicus, Vibrio hollisae, Vibrio fluvialis, Vibrio metchnikovii, Vibrio damsela and Vibrio furnisii), Yersinia sp. (such as Yersinia enterocolitica, Yersinia pestis, and Yersinia pseudotuberculosis) and Xanthomonas maltophilia among others.

In an exemplary embodiment, the primer can be designed for identification of Mycobacterium Tuberculosis (MTB). In certain embodiments, identification of drug resistance may be advantageous in large scale drug screening. By way of example, for MTB, primer design could include one or more of katG, 315ACC: Isoniazid resistance, rpoB, 531TTG: Rifampin resistance, gyrA, 94GGC: Fluoroquinolone resistance, and rrs, 1401G: Aminoglycoside resistance. Primers of signature genes related to MTB infection and TB symptoms can be utilized, and screening of treatments for both host and bacterial treatments can be effected by primer choice and design. Primers in certain cases can be utilized for capture along pathways that are turned on or off during infection, or activated/repressed in hosts with active infection. Particular pathways/markers that can be used in design of primers for tuberculosis can be found, for example in International Patent Application No. PCT/US18/56168, filed Oct. 16, 2018, at [0264], [0295] and Tables 1 and 2, incorporated herein by reference. The approach of utilizing primers in proximity or adjacent to a sequence of interest, which may include particular pathways implicated in an infection or disease can enrich for samples in larger drug scale drug screening, including for either host and/or microbial, e.g., bacterial treatments. Accordingly, primers for drug resistant strains, pathways of interest, families, classes or other groupings of interest can be utilized for enrichment, large scale drug studies, and other high throughput applications can be utilized. Primers adjacent to, or within about 100, about 90, about 80, about 70, about 60, about 50, about 40, about 30, about 20, abour 10, about 9, about 8, about 7, about 6, about 5, about 4, about 3, about 2, or about 1 nucleotide(s) of the gene of interest can be used.

Primers may be used to enrich for a viral infection (e.g. of a subject or plant), including a DNA virus, a RNA virus, or a retrovirus. Non-limiting example of viruses useful with the present invention include, but are not limited to Ebola, measles, SARS, Chikungunya, hepatitis, Marburg, yellow fever, MERS, Dengue, Lassa, influenza, rhabdovirus or HIV. A hepatitis virus may include hepatitis A, hepatitis B, or hepatitis C. An influenza virus may include, for example, influenza A or influenza B. An HIV may include HIV 1 or HIV 2. In certain example embodiments, the viral sequence may be a human respiratory syncytial virus, Sudan ebola virus, Bundibugyo virus, Tai Forest ebola virus, Reston ebola virus, Achimota, Aedes flavivirus, Aguacate virus, Akabane virus, Alethinophid reptarenavirus, Allpahuayo mammarenavirus, Amapari mmarenavirus, Andes virus, Apoi virus, Aravan virus, Aroa virus, Arumwot virus, Atlantic salmon paramyxovirus, Australian bat lyssavirus, Avian bornavirus, Avian metapneumovirus, Avian paramyxoviruses, penguin or Falkland Islandsvirus, BK polyomavirus, Bagaza virus, Banna virus, Bat herpesvirus, Bat sapovirus, Bear Canon mammarenavirus, Beilong virus, Betacoronavirus, Betapapillomavirus 1-6, Bhanja virus, Bokeloh bat lyssavirus, Borna disease virus, Bourbon virus, Bovine hepacivirus, Bovine parainfluenza virus 3, Bovine respiratory syncytial virus, Brazoran virus, Bunyamwera virus, Caliciviridae virus. California encephalitis virus, Candiru virus, Canine distemper virus, Canine pneumovirus, Cedar virus, Cell fusing agent virus, Cetacean morbillivirus, Chandipura virus, Chaoyang virus, Chapare mammarenavirus, Chikungunya virus, Colobus monkey papillomavirus, Colorado tick fever virus, Cowpox virus, Crimean-Congo hemorrhagic fever virus, Culex flavivirus, Cupixi mammarenavirus, Dengue virus, Dobrava-Belgrade virus, Donggang virus, Dugbe virus, Duvenhage virus, Eastern equine encephalitis virus, Entebbe bat virus, Enterovirus A-D, European bat lyssavirus 1-2, Eyach virus, Feline morbillivirus, Fer-de-Lance paramyxovirus, Fitzroy River virus, Flaviviridae virus, Flexal mammarenavirus, GB virus C, Gairo virus, Gemycircularvirus, Goose paramyxovirus SF02, Great Island virus, Guanarito mammarenavirus, Hantaan virus, Hantavirus Z10, Heartland virus, Hendra virus, Hepatitis A/B/C/E, Hepatitis delta virus, Human bocavirus, Human coronavirus, Human endogenous retrovirus K, Human enteric coronavirus, Human genital-associated circular DNA virus-1, Human herpesvirus 1-8, Human immunodeficiency virus 1/2, Human mastadenovirus A-G, Human papillomavirus, Human parainfluenza virus 1-4, Human paraechovirus, Human picornavirus, Human smacovirus, Ikoma lyssavirus, Ilheus virus, Influenza A-C, Ippy mammarenavirus, Irkut virus, J-virus, JC polyomavirus, Japanese encephalitis virus, Junin mammarenavirus, KI polyomavirus, Kadipiro virus, Kamiti River virus, Kedougou virus, Khuj and virus, Kokobera virus, Kyasanur forest disease virus, Lagos bat virus, Langat virus, Lassa mammarenavirus, Latino mammarenavirus, Leopards Hill virus, Liao ning virus, Ljungan virus, Lloviu virus, Louping ill virus, Lujo mammarenavirus, Luna mammarenavirus, Lunk virus, Lymphocytic choriomeningitis mammarenavirus, Lyssavirus Ozernoe, MSSI2\.225 virus, Machupo mammarenavirus, Mamastrovirus 1, Manzanilla virus, Mapuera virus, Marburg virus, Mayaro virus, Measles virus, Menangle virus, Mercadeo virus, Merkel cell polyomavirus, Middle East respiratory syndrome coronavirus, Mobala mammarenavirus, Modoc virus, Moijang virus, Mokolo virus, Monkeypox virus, Montana myotis leukoenchalitis virus, Mopeia lassa virus reassortant 29, Mopeia mammarenavirus, Morogoro virus, Mossman virus, Mumps virus, Murine pneumonia virus, Murray Valley encephalitis virus, Nariva virus, Newcastle disease virus, Nipah virus, Norwalk virus, Norway rat hepacivirus, Ntaya virus, O′nyong-nyong virus, Oliveros mammarenavirus, Omsk hemorrhagic fever virus, Oropouche virus, Parainfluenza virus 5, Parana mammarenavirus, Parramatta River virus, Peste-des-petits-ruminants virus, Pichande mammarenavirus, Picornaviridae virus, Pirital mammarenavirus, Piscihepevirus A, Porcine parainfluenza virus 1, porcine rubulavirus, Powassan virus, Primate T-lymphotropic virus 1-2, Primate erythroparvovirus 1, Punta Toro virus, Puumala virus, Quang Binh virus, Rabies virus, Razdan virus, Reptile bornavirus 1, Rhinovirus A-B, Rift Valley fever virus, Rinderpest virus, Rio Bravo virus, Rodent Torque Teno virus, Rodent hepacivirus, Ross River virus, Rotavirus A-I, Royal Farm virus, Rubella virus, Sabia mammarenavirus, Salem virus, Sandfly fever Naples virus, Sandfly fever Sicilian virus, Sapporo virus, Sathuperi virus, Seal anellovirus, Semliki Forest virus, Sendai virus, Seoul virus, Sepik virus, Severe acute respiratory syndrome-related coronavirus, Severe fever with thrombocytopenia syndrome virus, Shamonda virus, Shimoni bat virus, Shuni virus, Simbu virus, Simian torque teno virus, Simian virus 40-41, Sin Nombre virus, Sindbis virus, Small anellovirus, Sosuga virus, Spanish goat encephalitis virus, Spondweni virus, St. Louis encephalitis virus, Sunshine virus, TTV-like mini virus, Tacaribe mammarenavirus, Taila virus, Tamana bat virus, Tamiami mammarenavirus, Tembusu virus, Thogoto virus, Thottapalayam virus, Tick-borne encephalitis virus, Tioman virus, Togaviridae virus, Torque teno canis virus, Torque teno douroucouli virus, Torque teno felis virus, Torque teno midi virus, Torque teno sus virus, Torque teno tamarin virus, Torque teno virus, Torque teno zalophus virus, Tuhoko virus, Tula virus, Tupaia paramyxovirus, Usutu virus, Uukuniemi virus, Vaccinia virus, Variola virus, Venezuelan equine encephalitis virus, Vesicular stomatitis Indiana virus, WU Polyomavirus, Wesselsbron virus, West Caucasian bat virus, West Nile virus, Western equine encephalitis virus, Whitewater Arroyo mammarenavirus, Yellow fever virus, Yokose virus, Yug Bogdanovac virus, Zaire ebolavirus, Zika virus, or Zygosaccharomyces bailii virus Z viral sequence. Examples of RNA viruses for which primers may be designed include one or more of (or any combination of) Coronaviridae virus, a Picornaviridae virus, a Caliciviridae virus, a Flaviviridae virus, a Togaviridae virus, a Bornaviridae, a Filoviridae, a Paramyxoviridae, a Pneumoviridae, a Rhabdoviridae, an Arenaviridae, a Bunyaviridae, an Orthomyxoviridae, or a Deltavirus. In certain example embodiments, the virus is Coronavirus, SARS, Poliovirus, Rhinovirus, Hepatitis A, Norwalk virus, Yellow fever virus, West Nile virus, Hepatitis C virus, Dengue fever virus, Zika virus, Rubella virus, Ross River virus, Sindbis virus, Chikungunya virus, Borna disease virus, Ebola virus, Marburg virus, Measles virus, Mumps virus, Nipah virus, Hendra virus, Newcastle disease virus, Human respiratory syncytial virus, Rabies virus, Lassa virus, Hantavirus, Crimean-Congo hemorrhagic fever virus, Influenza, or Hepatitis D virus.

A nucleic acid identifier can further include a unique molecular identifier and/or additional barcodes specific to, for example, a common support to which one or more of the nucleic acid identifiers are attached. Thus, a pool of target molecules can be added, for example, to a discrete volume containing multiple solid or semisolid supports (for example, beads) representing distinct treatment conditions (and/or, for example, one or more additional solid or semisolid support can be added to the discreet volume sequentially after introduction of the target molecule pool), such that the precise combination of conditions to which a given target molecule was exposed can be subsequently determined by sequencing the unique molecular identifiers associated with it.

Labeled target molecules and/or target nucleic acids associated origin-specific nucleic acid barcodes (optionally in combination with other nucleic acid barcodes as described herein) can be amplified by methods known in the art, such as polymerase chain reaction (PCR). For example, the nucleic acid barcode can contain universal primer recognition sequences that can be bound by a PCR primer for PCR amplification and subsequent high-throughput sequencing. In certain embodiments, the nucleic acid barcode includes or is linked to sequencing adapters (for example, universal primer recognition sequences) such that the barcode and sequencing adapter elements are both coupled to the target molecule. In particular examples, the sequence of the origin specific barcode is amplified, for example using PCR. In some embodiments, an origin-specific barcode further comprises a sequencing adaptor. In some embodiments, an origin-specific barcode further comprises universal priming sites. A nucleic acid barcode (or a concatemer thereof), a target nucleic acid molecule (for example, a DNA or RNA molecule), a nucleic acid encoding a target peptide or polypeptide, and/or a nucleic acid encoding a specific binding agent may be optionally sequenced by any method known in the art, for example, methods of high-throughput sequencing, also known as next generation sequencing or deep sequencing. A nucleic acid target molecule labeled with a barcode (for example, an origin-specific barcode) can be sequenced with the barcode to produce a single read and/or contig containing the sequence, or portions thereof, of both the target molecule and the barcode. Exemplary next generation sequencing technologies include, for example, Illumina sequencing, Ion Torrent sequencing, 454 sequencing, SOLiD sequencing, and nanopore sequencing amongst others. In some embodiments, the sequence of labeled target molecules is determined by non-sequencing-based methods. For example, variable length probes or primers can be used to distinguish barcodes (for example, origin-specific barcodes, for instance well barcode or cell barcode) labeling distinct target molecules by, for example, the length of the barcodes, the length of target nucleic acids, or the length of nucleic acids encoding target polypeptides. In other instances, barcodes can include sequences identifying, for example, the type of molecule for a particular target molecule (for example, polypeptide, nucleic acid, small molecule, or lipid). For example, in a pool of labeled target molecules containing multiple types of target molecules, polypeptide target molecules can receive one identifying sequence, while target nucleic acid molecules can receive a different identifying sequence. Such identifying sequences can be used to selectively amplify barcodes labeling particular types of target molecules, for example, by using PCR primers specific to identifying sequences specific to particular types of target molecules. For example, barcodes labeling polypeptide target molecules can be selectively amplified from a pool, thereby retrieving only the barcodes from the polypeptide subset of the target molecule pool. Barcodes for population-based applications, for example populations of cells, can be identified by a well barcode. In certain instances, the engineered well identification barcode should be sufficiently perpendicular to be able to be differentiated for demultiplexing. In an exemplary environment, the population beads comprise a PCR handle, well identification barcode and a unique molecular identifier.

A nucleic acid barcode can be sequenced, for example, after cleavage, to determine the presence, quantity, or other feature of the target molecule. In certain embodiments, a nucleic acid barcode can be further attached to a further nucleic acid barcode. For example, a nucleic acid barcode can be cleaved from a specific-binding agent after the specific-binding agent binds to a target molecule or a tag (for example, an encoded polypeptide identifier element cleaved from a target molecule), and then the nucleic acid barcode can be ligated to an origin-specific barcode. The resultant nucleic acid barcode concatemer can be pooled with other such concatemers and sequenced. The sequencing reads can be used to identify which target molecules were originally present in which discrete volumes.

In one aspect, the bead can be functionalized with a TCR specific sequence. As an example, VDJdb is a comprehensive database of antigen-specific T-cell receptor (TCR) sequences. Shugay et al. Nucleic Acids Research, Volume 46, Issue D1, 4 Jan. 2018, Pages D419-D427, DOI:10.1093/nar/gkx760

In an embodiment, the solid support comprises a bead or a population of beads, or a micro-bead or a population of micro-beads, micro-arrays, micro-wells, or micro-lids. In some aspect, the solid support comprises a hydrogel bead or a magnetic bead or a magnetic core. In an aspect of the embodiment, the solid support is a silica bead, a cellulose bead, or an agarose bead. In another aspect of the embodiment, the bead has a shape that is circular, square, star, or porous.

In an embodiment, the solid support comprises a polymer. In an aspect of the embodiment, the solid support comprises hydroxylated methacrylic polymer or hydroxylated poly(methyl methacrylate). In another aspect of the embodiment, the solid support comprises polystyrene polymer, polypropylene polymer, polyethylene polymer, agarose, or cellulose.

In an embodiment of any of the above method or composition, the solid support has an average particle size ranging from 10 microns to 500 microns. Length of spacers can be tuned based on the size of the solid support.

In particular embodiments, the solid support has an average particle size between about 10 microns to 200 microns, about 10 microns to 190 microns, about 10 microns to 180 microns, about 10 microns to 170 microns, about 10 microns to 160 microns, about 10 microns to 150 microns, about 10 microns to about 140 microns, about 10 to about 130 microns, about 10 to about 120 microns, about 10 microns to about 110 microns, about 10 microns to about 100 microns, about 10 microns to about 90 microns, about 10 microns to about 80 microns, about 10 microns to about 70 microns, about 10 microns to about 60 microns, about 10 microns to about 50 microns, about 10 microns to about 40 microns, about 10 microns to 30 microns, about 10 microns to about 20 microns, about 20 microns to about 30 microns, about 20 microns to about 40 microns, about 20 microns to about 50 microns, about 20 microns to about 60 microns, about 20 microns to about 70 microns, about 20 microns to about 80 microns, about 20 microns to about 100 microns, about 20 microns to about 100 microns, about 50 microns to about 100 microns, about 100 microns to 200 microns, or about 30 microns. In some embodiments, the bead or micro-bead has an average size, measured as average diameter of 20-40 μm.

The solid support is preferably provided functionalized. The solid support may be functionalized to permit covalent attachment of the agent and/or label. Such functionalization on the support may comprise reactive groups that permit covalent attachment to an agent and/or a label. In embodiments, the solid support is functionalized with a spacer and surface reactive nucleotide that can be further reacted with nucleotides, oligonucleotides, barcodes and the like. The surface reactive nucleotide provided on the solid support for further reacting can be a nucleotide that comprises part of a sequence for a PCR handle or other component of the nucleic acid sequence appended to the solid support.

A solid support can mean a bead or micro-bead, or a plurality of micro-beads, micro-arrays, micro-wells, or micro-lids. The solid support can be shaped in any manner required for an end use application and may have a shape that is circular, square, star, or porous. Examples of suitable solid supports include, but are not limited to, inert polymers (preferably non-nucleic acid polymers), beads, glass, or peptides. In some embodiments, the solid support is an inert polymer or a bead. The bead is a silica bead, a hydrogel bead or a magnetic bead. In some embodiments, the solid support comprises a magnetic core.

Deprotecting

In embodiments, the protecting groups of reactive nucleotides are subjected to a step of deprotecting. Deprotecting of the nucleic acid molecules in preferred embodiments is accomplished in ammonia hydroxide. In particular embodiments, the deprotecting is performed in 20% to about 33% ammonia hydroxide. In embodiments, 25% to 33%, 26%, 27%, 28%, 29%, 30%, 31%, 32%, 33%, ammonia hydroxide, in certain embodiments in 30% ammonia hydroxide.

In certain embodiments, the deprotecting is performed at a temperature of about 15° C. to about 30° C., about 18° C. to about 27° C., about 20° C. to about 25° C. , or at about room temperature.

In particular embodiments, the deprotecting step is performed for about 30 minutes, 40 minutes, 50 minutes, 60 minutes, 70 minutes, 80 minutes, 90 minutes, 100 minutes, 110 minutes, 120 minutes, 130 minutes, 140 minutes, 150 minutes, or about 180 minutes.

A more effective or enhanced process may comprise improved retention of oligonucleotides, reduced shedding or loss of oligonucleotides, fewer blank solid supports, and/or increased viability of the solid supports after completion of the methods.

### Methods

Methods of detecting the presence of functionalization on a solid support are provided, and comprise contacting the solid support with a fluorescent probe. In some embodiments, more than one probe can be used to look at oligosynthesis efficiency. The fluorescent probe can comprise an oligonucleotide capable of binding moieties when present on the solid support. The moieties may be, for example, any consistent sequence on a bead. In an aspect, the sequence is a capture sequence, universal handle, PCR or other sequence that is consistent in each bead.

In embodiments, the assays are designed to measure fluorescence, the levels of fluorescence reflecting stoichiometric presence of functionalization of beads. In one aspect, the method comprises quantifying the amount of functionalization of the solid support. Quantifying can comprise measuring an amount of fluorescence and can comprise comparing to baseline, controls, or other known functionalized beads. In particular embodiments, fluorescent sorting of beads using pre-defined sorting gates can aid in evaluating quality of beads. Pre-defined sorting gates can be established using a control bead, commercially available bead, or other gating In embodiments, beads are further sorted by high probe content. In an exemplary approach, beads with a capture region, such as poly-A capture region, can be bound with a fluorescently labeled DNA probe that binds the poly-A capture region. A gate can be established to sort based on where the majority of beads sit. Additional gates can be establishing to show beads comprising a large amount of functionality, e.g. high poly-T content in this example. In certain embodiments, the beads can be sorted with the probes removed and used after sorting. In one aspect, the beads can be sorted according to quality, quantity of functionalization according to gating. In certain embodiments, probes or batches of probes identified as comprising more than desired amounts of oligonucleotides on the beads, which can advantageously be processed to remove additional oligonucleotides. Oligo density can be evaluated based on distinct size gates as described herein. Further optimization of annealing conditions for preparation of the functionalized solid support can be provided by gating analysis of solid supports using varying reaction conditions. In an aspect, extension reaction optimization can further be optimized, utilizing varying reaction volume and extension handle concentration. In certain embodiments, by use of photocleavable linkages, the exposure to appropriate wavelength of light for the linkers can remove oligonucleotides.

The present invention provides a method for characterizing a functionalized solid support having a spacer, wherein the method comprises cleaving a linkage located between the spacer and the solid support, whereby the spacer is detached from the solid support. In some aspects, the linkage comprises a disulfide linkage, a photolabile linker, a halide ion labile linker, or any cleavable linker, the examples of which are described for the spacer herein. In one embodiment, the method comprises isolating the spacer. In another embodiment, the method comprises determining the mass or molecular weight of the spacer, e.g., via mass spectroscopy, chromatography, etc. In one embodiment, the method is used to validate a method for functionalizing the solid support. Additionally, in this manner, the oligonucleotides that are shed by cleavage of the linker can also be examined for quality by gel assay and/or other sequencing methods. Upon determination that the solid support functionalization is of the quantity and quality desired, e.g. for example, after sorting, the probes can be removed using basic conditions, e.g. treatment with solution having a pH >7.5, 8.0, 8.5, 9.0, 9.5, 10.0.

Assay optimization can include identifying the salt concentration required for probe binding for a given probe sequence and may comprise suspension in a 0.8 M, 1.0 M. 1.2 M, 1.4 M or more NaCl or other salt solution. The amount of probe needed to saturate the surface for a particular bead batch can also be optimized.

The invention provides for a method for nucleic acid analysis using the solid support described herein. The method for nucleic acid analysis can comprise single cell analysis, RNA analysis, DNA analysis, chromatin analysis, RNA-Seq, or bulk population analysis when population beads are utilized. The method can comprise processing an analyte comprising a protein, a peptide, an antibody, an organelle, a cell, a cellular fraction, or processing a clinical sample. The method can comprise single cell microfluidics analysis or Drop-Seq (see International Patent Publication No. WO2016/040476) or single cell microwell analysis (see International Patent Publication No. WO/2017124101 and Jinzhou Yuan & Peter A. Sims, An Automated Microwell Platform for Large-Scale Single Cell RNA-Seq, Scientific Reports 6, 33883 (2016), doi:10.1038/srep33883).

### Population Beads

Methods of using functionalized solid supports that in high-throughput RNA sequencing are provided, In certain embodiments, the functionalized solid supports described herein are prepared for use as population beads . In certain instances, a plurality of solid supports is provided, the solid supports comprising the well barcode, primers, a unique molecular identifier, and an oligo-dT and/or capture oligonucleotide, with the high-throughput method further comprising pooling beads in an individual discrete volume, seeding cells in the individual discrete volume, and conducting high throughput RNA sequencing for a population of cells in each individual discrete volume. In an embodiment, methods of compressive sensing for ultra-low depth sequencing can be utilized with population beads. See, e.g., Cleary et al., “Efficient Generation of Transcriptomic Profiles by Random Composite Measurements.” DOI:10.1016/j .cell.2017.10.023. In particular embodiments, population beads can be utilized in organoid screening, for example identification of changes in composition or function with specific cell types. In one aspect, module score enrichments for individual cell type identities can be used as an approach. The approaches of population bead methodologies described herein can provide improvement over recent primer-based approaches for scalable RNA-seq. Without being bound by theory, the advantages of these methods provide the ability not only to apply bead-based analysis to bulk RNA-seq, but to provide with a high throughput similar to the throughput provided in single cell sequencing space. In particular embodiments, the bead sets can be pooled before reverse transcription, after reverse transcription, before second strand sequencing, after second strand sequencing, before whole transcriptome amplification, or after whole transcriptome amplification. In particular embodiments, tagmenting is performed after second strand sequencing. In certain embodiments, the plurality of solid supports can comprise the well barcode, a primer, a unique molecular identifier, and an oligo-dT and/or capture oligonucleotide. In certain embodiments, the method can further comprise the steps of pooling beads in an individual discrete volume, seeding cells in the individual discrete volume, and conducting high throughput RNA sequencing for a population of cells in each individual discrete volume. In one aspect, the well barcode comprises about 4 nucleotides, about 5 nucleotides, about 6 nucleotides, about 7 nucleotides, about 8 nucleotides, about 9 nucleotides, or about 10 nucleotides, in certain embodiments 6 nucleotides. In certain embodiments, the UMI comprises about 10 to about 20 nucleotides , in certain embodiments, about 14 oligonucleotides.

The invention is further described in the following examples, which do not limit the scope of the invention described in the claims.

### EXAMPLES

### Example 1

Applicants have explored methods to improve solid support functionalization. Differentiators with current commercially available beads are provided below and include UMI complexity, number of viable beads, cell barcode complexity. As discussed further in the examples, Applicants have sought to develop an assay to further probe quality of the functionalized beads prepared according the current methods versus commercially available beads.

UMI complexity will be determined by sequencing. As it stands now, Applicants see a large enrichment of guanine on commercially available beads. Applicants anticipate a more even distribution of bases in UMI sequences prepared according to currently disclosed methods.

Number of viable beads have improved with Applicant developed methods. Based on probe sorting, about 10% of commercially available beads Applicants evaluated as not viable (not enough oligo strands on them). The bead lots utilizing the current methods have shown fewer blank beads in limited syntheses. It is possible this is stemming from Applicants' deprotection protocol. Applicants are deprotecting at room temperature for one hour in 30% ammonium hydroxide. Typically, this deprotection is done at 80C, but seeing oligo shedding was seen with heat as compared to deprotecting at room temperature. Increased coupling times may also improve bead viability.

Cell barcode complexity improvements are anticipated. The split-and-pool step is a likely area for significant degradation and will be tested with probes using the quality control assays developed herein to be certain. Next, Applicants will consider moving towards modifying the split-and-pool protocol to minimize the number of times beads need to be removed from the instrument. This is potentially tied to diminished bead viability.

Number of capture sites appear to be improved using current methodology. However, more testing with the probe assays for quality control and ultimately looking at a few sequencing runs will be performed.

Probe quality control measure: Applicants advantageously can quickly identify poor syntheses using a fluorescent oligo probe. Eventually, Applicants may be able to sort beads with high probe content to guarantee only high-quality beads are used for SeqWell.

Multisequence capture design is anticipated. In addition to poly-T capture baits, multisequence capture, including randomers can be used. Solid supports that are partially poly-T and partially enriching for other sequences are planned. These sequences are referred to as universal barcodes, or a sequence that could then be extended off of using stoichiometrically mixed primers analogous to Applicants' sequences of interest. As described herein, Applicants would adapt methods developed in Han et al., Cell, 172:5, p. 1091-1107 at Fig. S1a (2018) or one being developed in-house. Sequencing data from SeqWell beads will be utilized to determine best approach methodology. Paired Sequence enrichment sequencing using Poly T and Capture Baits, such as for HIV are envisioned. Following a method similar to Han for barcoding, Applicants can stoichiometrically spiking in capture baits, and can have probes specific to poly T and also compared to capture and quantitative and set gates for sorting.

Engineered barcodes: In Han et al. an extension method is used to stitch together three premade barcodes. While the approach avoids the split-and-pool, a potential loss point, it may require more sequencing reads and is likely less time and cost efficient. This method will be explored and possibly adapted depending on efficiency, see, Han et al. at section entitled “Synthesis of barcoded beads” the method incorporated herein by reference specifically. Using pools with pairs or triplets is one approach, preventing time when beads not in inert atmosphere and transferred from vessels, likely contributing factor to bead loss and loss of quality (including increased truncations).

Use of smaller beads are envisioned, including placing oligos on smaller beads that are chemically similar to hydroxylated methacrylate beads that are commercially available. Use of smaller beads would allow use in smaller wells, allowing more wells on an array. When using a smaller or microbead, the spacer length may be adjusted and tuned to provide the most desirable properties, including reducing spacer length to adjust for the smaller bead size.

### Example 2

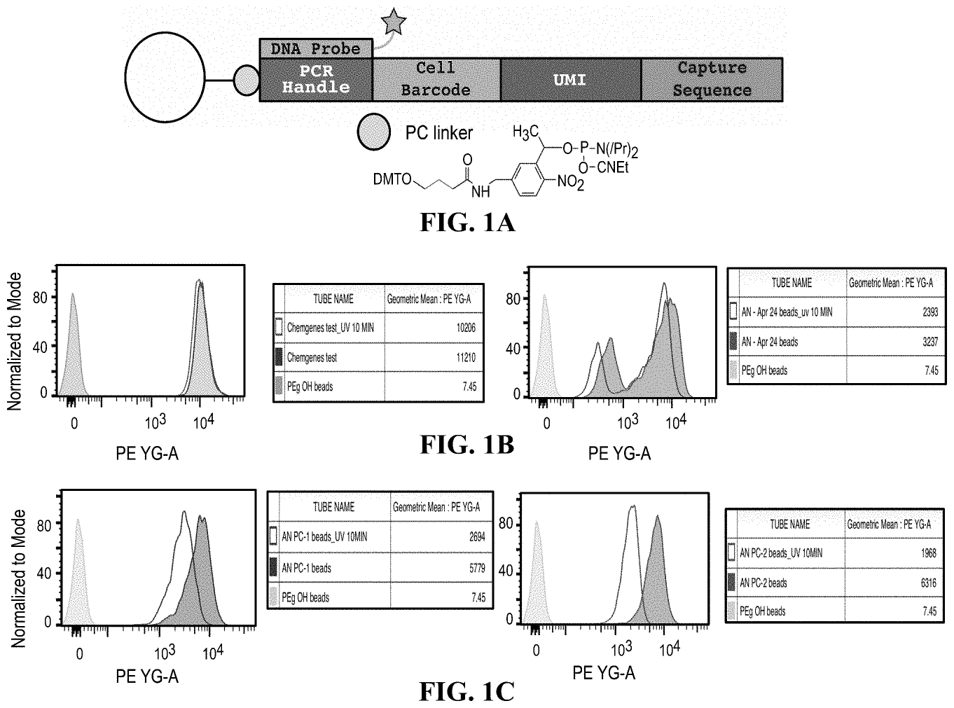

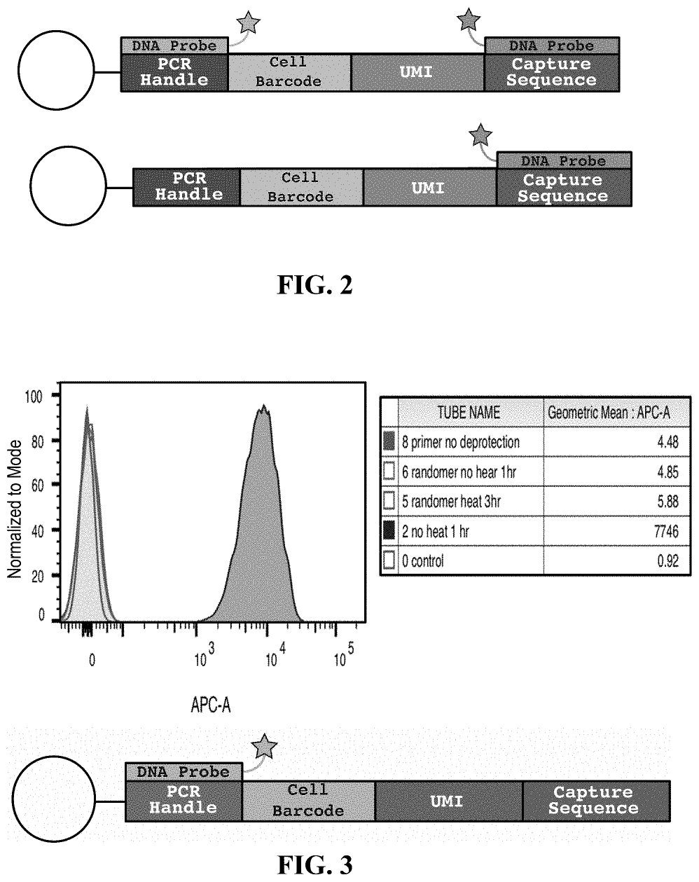

Using FIG. 1A for reference of an exemplary solid support, and working from inside/out, PEG is used as an exemplary spacer to move the DNA off the bead itself if longer on the new beads, and so sort of stimulating a non-solid state, preferably using closer to 5, 10, 18 repeats of PEG, focusing on 2K. a PEG spacer length longer than 18 repeats, smaller than 150 gives behavior that approximates non-solid state. In certain embodiments, a 2k-10k PEG spacer is planned. Commercially available solid supports use a spacer length akin to 150 repeats where the spacer turns from repeats to mass, using a spacer on the order of kiloDalton.

### Example 3

Method: A 1:50 dilution of stock probe in 1M NaCl (dissolved in TE) is made. A 100 uL portion of the above solution is mixed with 25k beads. The solution is mixed for 30 minutes before the beads are washed and suspended in 1M NaCl, at which point the beads can be sorted. Note, salt concentration varies based on probe sequence. Multiple probes can be used to determine oligosynthesis efficiency. Beads can be sorted and probe can be removed and used after sorting. While conditions vary based on the probe sequence used, generally basic conditions remove the probe.

Quality control of commercially available beads indicate photocleavable linkers with quite a bit of drop off with barcoding. Commercially available beads were shown to contain UMIs massively increased for guanine. Also, interesting in using probe, ˜10% of the commercially available beads do not have oligos.

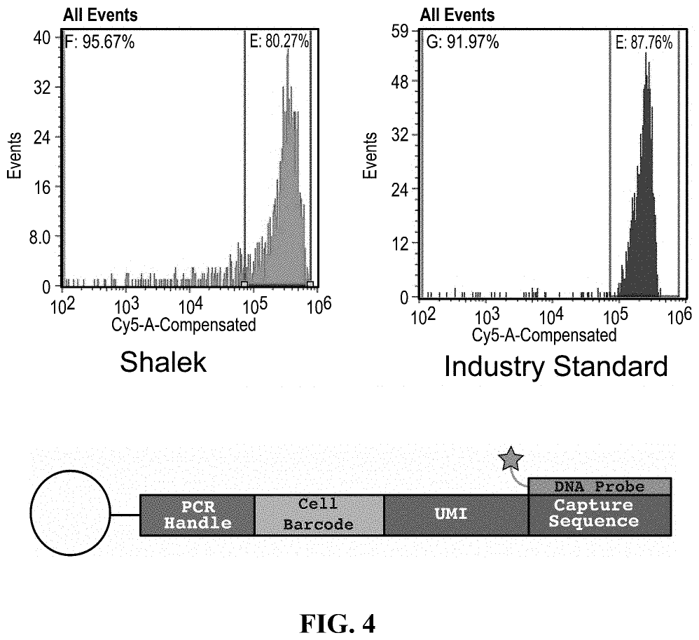

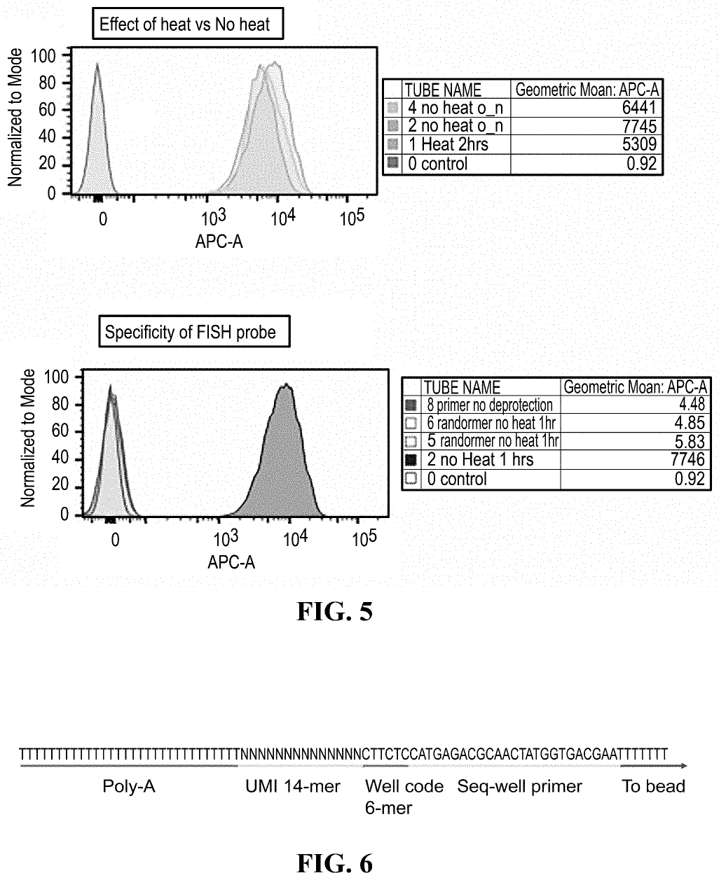

Using same gating, Applicants methods yield ˜95% or more, and could be higher with probe annealing tweaks, give melting point of probes. (FIG. 4).

### Example 4

Goals in the current example include preparation of solid supports in organoid screening for identifying changes in compositions, and function within specific cell types. For example, hits can be defined based on module score enrichments for individual cell type identities. Minimally-biased read-out for screening would also be advantageous to obtain a large amount of information for lower cost. The challenge in part is to employ methods of compressive sensing for ultra-low depth sequencing with meaningful interpretation, see, e.g. Cleary et al., “Efficient Generation of Transcriptomic Profiles by Random Composite Measurements.” DOI:10.1016/j .ce11.2017.10.023

Recent primer-based approaches for scalable RNA-seq either collapse post-RT (Ye et al, DOI:10.1038/s41467-018-06500-x or still require labor-intensive RNA-isolation steps. Alpern et al, BRB-seq: ultra-affordable high-throughput transcriptomics enabled by bulk RNA barcoding and sequencing.

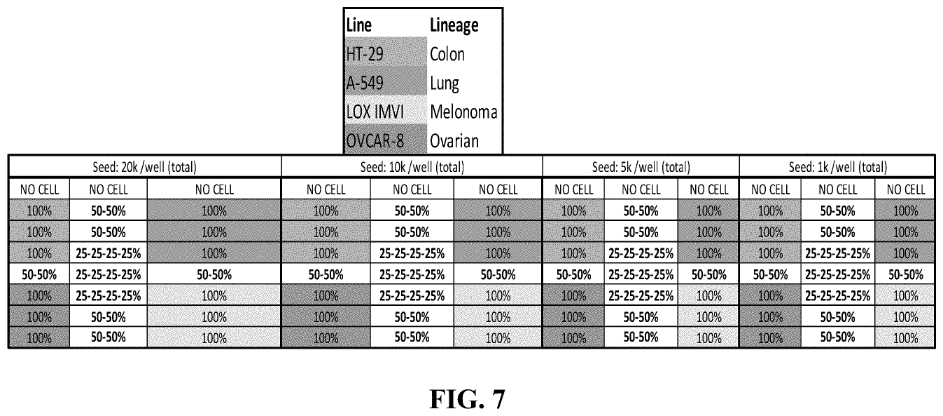

Population beads (FIG. 6) were prepared to include 96 6-mer barcodes engineered with ‘DNA barcodes’ R software package that generated a DNA barcode set using a minimal sequence level distance=2, using ashlock heuristic method. An initial pool of 24 well-coded beads were used in the pilot. In round 1 of the pilot (FIG. 7), frozen lysates of cell lines (30 uL) were added to beads (1 k/w) in hybridization (120 uL)/mix for 1 hour, and washed with hyb. The pool was 24 into 1. Wash 1x maxima, RT (v3 s-w), Exo +S3. Next WTA (v3 s-w, 24/tube). Nextera (3/tube)+single index (1/tube). Run on 1 Next-seq: target ˜2.5 mil mapped/sample.

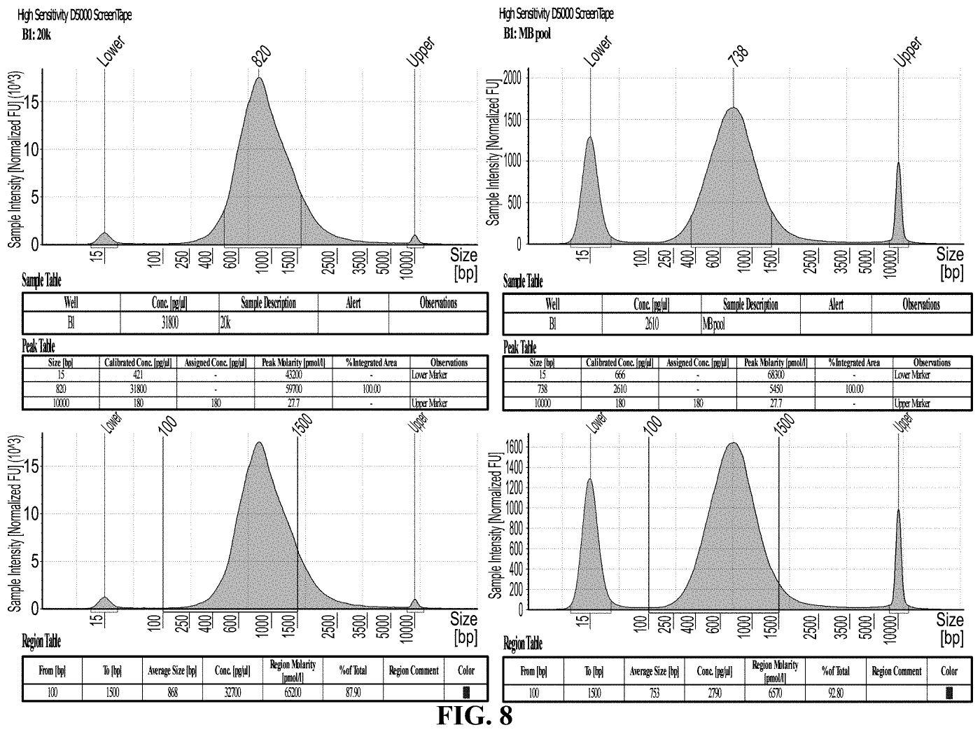

Results on WTA+Nextera+Next Seq are provided in FIG. 8. Nextera is a bit big. WAT PCR: 9 cycles (PCR13), with Nextera PCR: 12 cycles. NextSeq: loaded @2.2 pM.

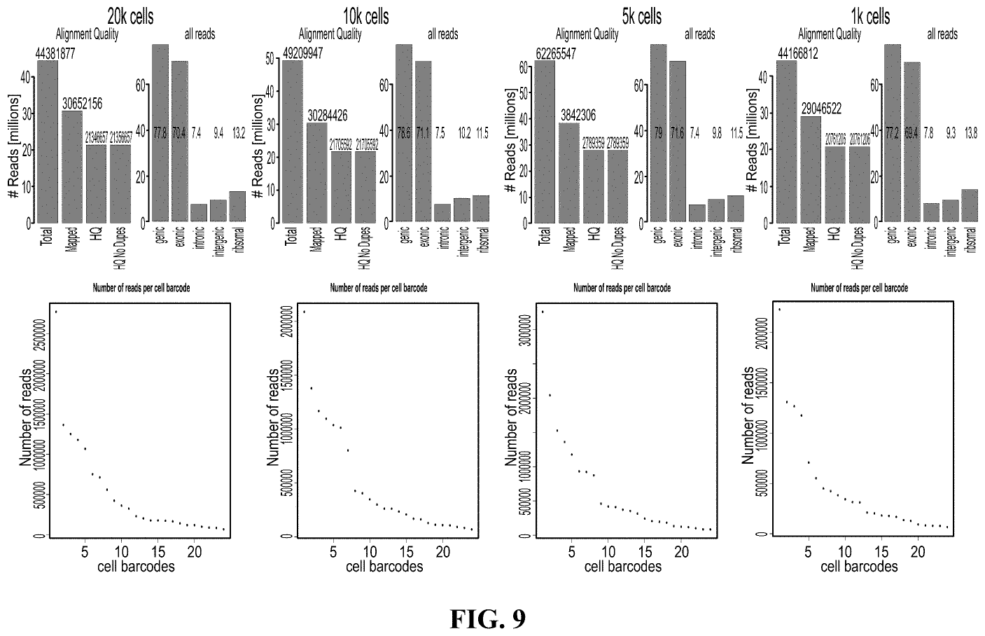

As shown in FIG. 9, an alignment with DropSeq pipeline included adjustment of barcode+UMI length, force cell count+feed in specified barcodes for alignment.

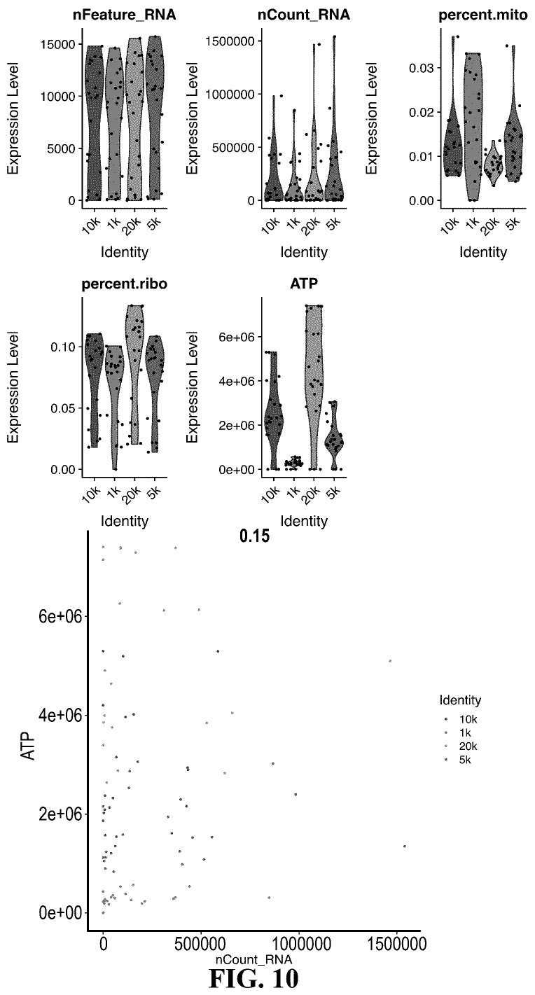

FIG. 10 charts show nUMI is independent of Cell number.

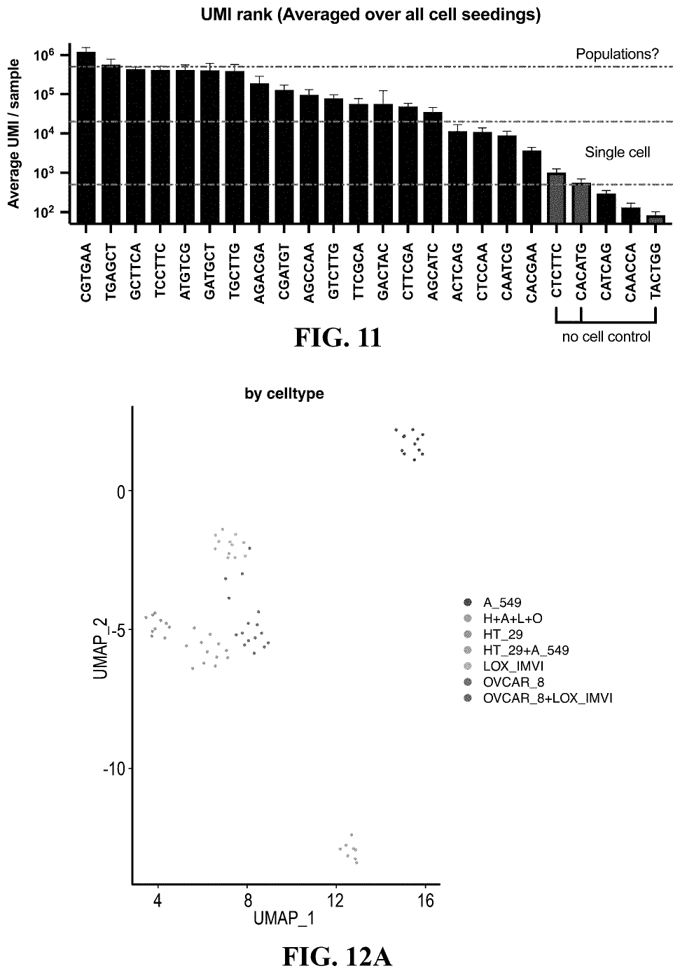

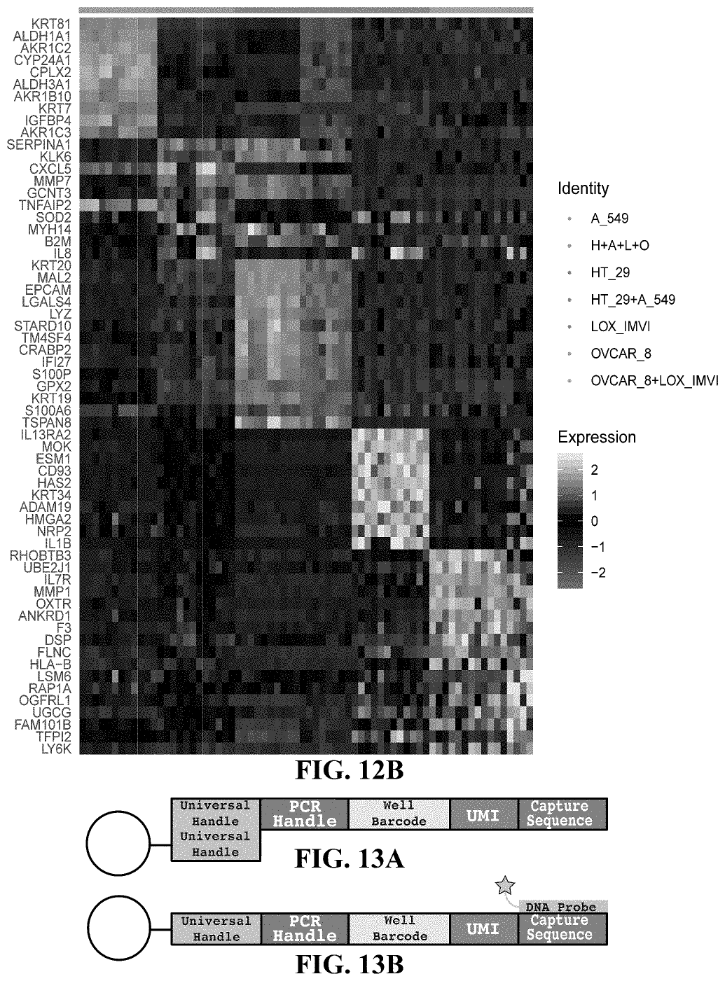

FIG. 11 looks at barcodes, reviewing the range of average UMI/sample around 104. Samples with UMI greater than 5000 are shown by cell type (FIG. 12A) and identity (FIG. 12B).

Bead synthesis challenges include linker conjugation and synthesizer issues. Changing up the bead sequence to switch the tail of UMI to only V is one possibility. Similarly, addition of VN at end of poly-T will be considered. Increase of minimal hamming distribution for cell barcodes will also be explored.

Regarding protocol optimization, because populations are not single cells, elements of protocol to consider changing may include the following parameters: pooling bead sets before versus after reverse transcription, before or after sss, and/or before or after WTA. Protocol optimization may also include significant reduction of WTA cycle number or tagmentation straight from single strand synthesis (SSS). Upon alignment and analysis, populations may require adjustments. For example, DropSeq tools has a ‘whitelist’ feature that only returns specified barcodes (akin to forcing). DropEst whitelist looks for specified barcodes and others, but stranded-ness is still an issue. A special workflow analysis will be developed to take into account use in populations.

### Example 5

DNA probes Applicants have proposed can biding any consistent sequence on the bead. Advantageously, the probe binds specifically to it's complement sequence and will not bind unprocessed oligos. The planned probes do not bind randomer sequences or sequences that are not deprotected. The probes can be configured to bind the PCR handle of the bead. FIG. 13A-13B shows templated synthesis off a shorter synthesized handle, with Klenow extension creating the final capture bead. Briefly, synthesis can comprise providing a solid support with a universal handle. Oligosynthesis can proceed with the addition of PCR Handle, Well barcode, UMI, and capture sequence. After oligosynthesis, a well specific sequence can be added. A Klenow extension can create the final capture bead. Quality control can be performed as described previously, by establishing a gating procedure to sort highly functionalized beads.

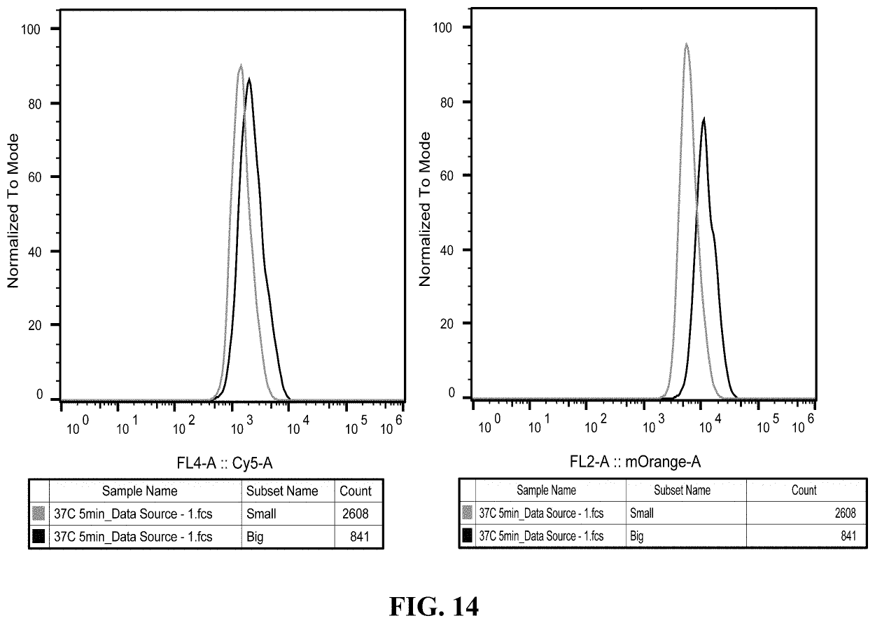

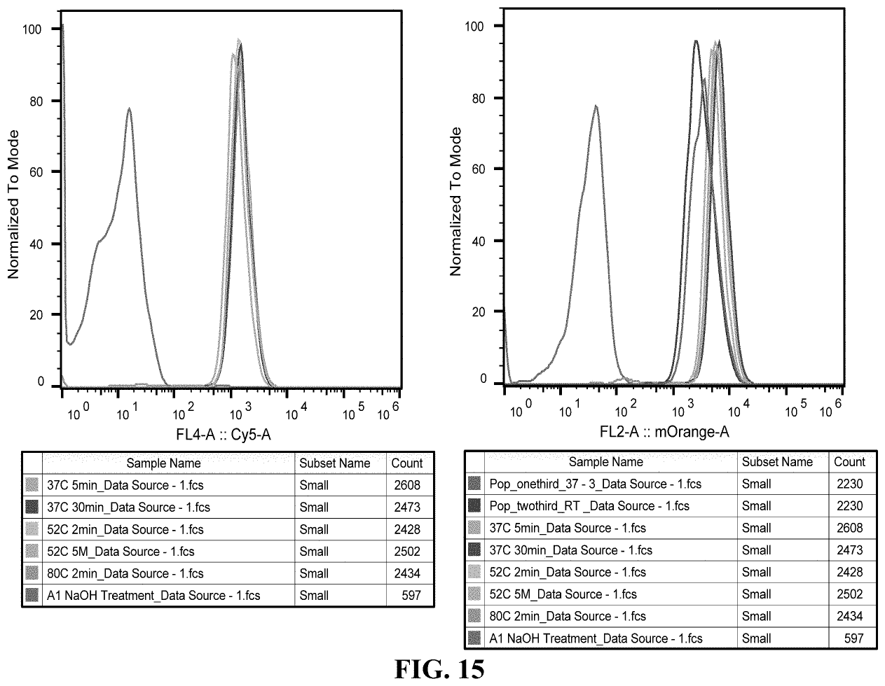

Fluorescently Activated Cell Sorting (FACS) analysis of different conditions were examined using the Applicant designed probes. Oligo density was analyzed based on distinct size gates were evaluated (FIG. 14). Different Annealing conditions of oligosynthesis of the nucleic acid sequence extended on the universal handle was evaluated. (FIG. 15). Based on analysis, probe annealing is effective across 37C-80C and 2 minutes to 5 minutes after reviewing varied temperature of annealing and time at temperature. Optimal reaction conditions according to evaluation are around 37C and 30 minutes.

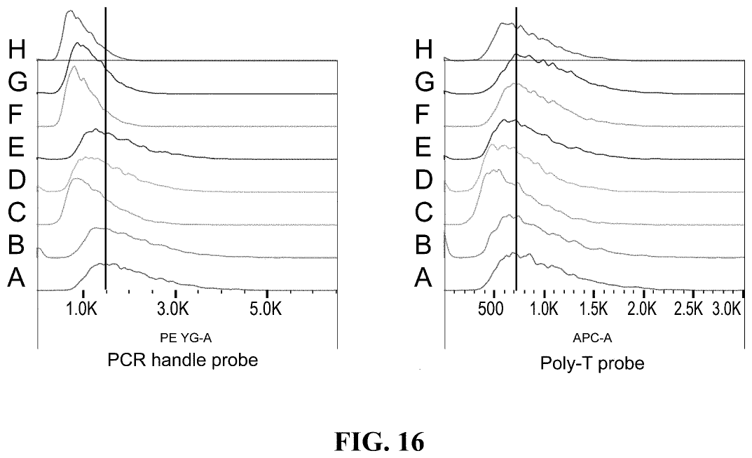

Extension reaction optimization was performed, showing extension reaction is not significantly limited by reaction volume (dTs and Klenow enzyme) (FIG. 16, Table 1). There is sight effect of extension handle concentration, suggesting the primary limiting process is synthesis of accessible linkage on bead surface.

TABLE 1
Extension reaction optimization

Extension rxn volume per beads

| | | 2.5 uL: 1k | 5 uL: 1k | 10 uL: 1k |
|---|---|---|---|---|
| | | beads | beads | beads |
| Conc, of | 15 uM | A | B | C |
| extension | 30 uM | | D | |
| handle | 60 uM | | E | |
| | 100 uM | F | G | H |

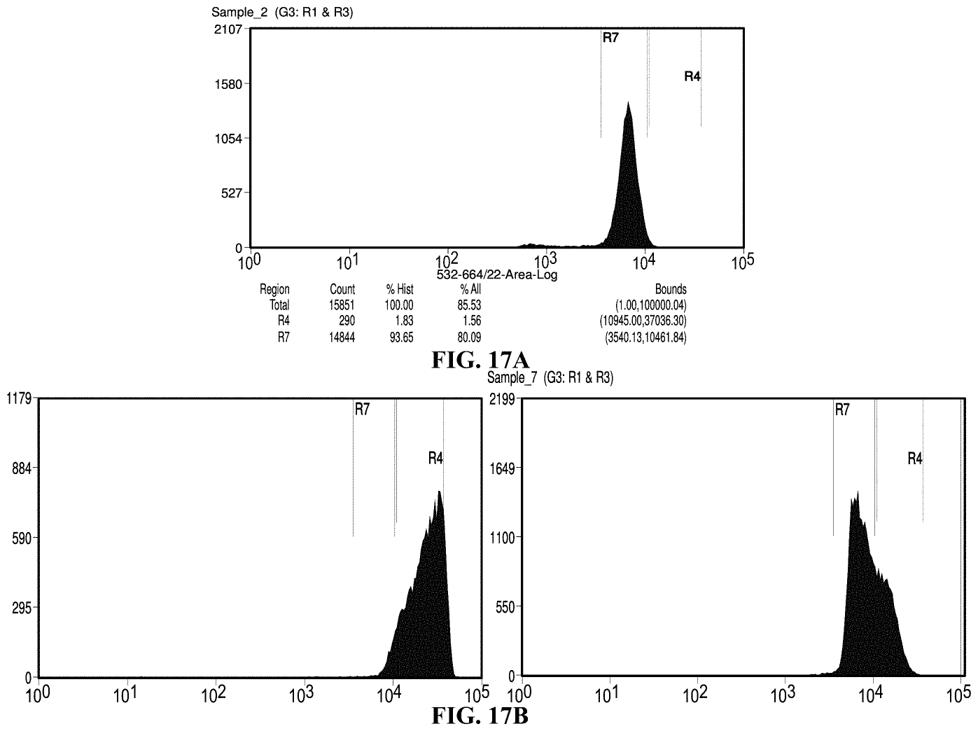

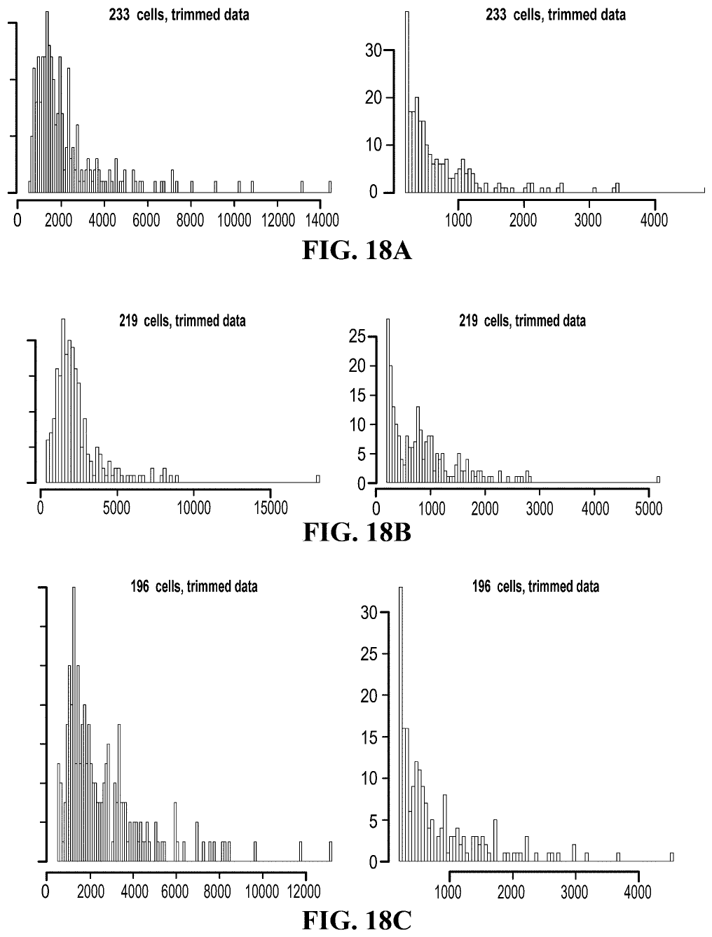

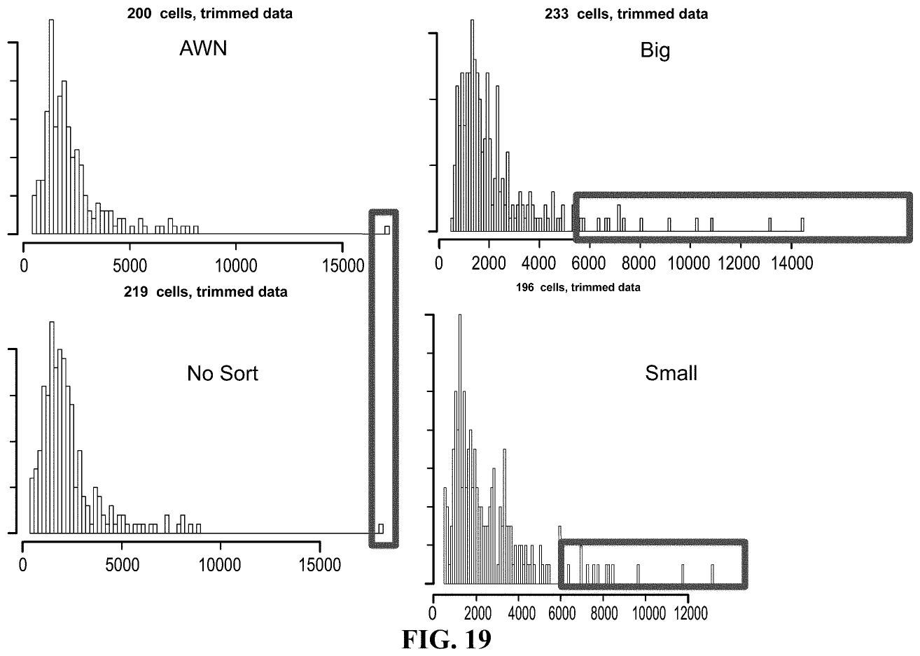

Single-Cell data showing improved oligosynthesis efficiency is shown in FIGS. 17A-1 B, comparing commercially available ChemGene beads (17A) and Applicant beads (17B). Commercially available beads were flow sorted first to define the gate (17A), with Applicant beads sorted second. FIG. 17B, left, shows initial sort of the beads; FIG. 17B, right, shows beads being sorted after 6-7 hours (not on ice). From FIG. 17B clearly indicates probe shedding. Histograms of transcript and gene count from gates are shown in FIGS. 18A-18C. FIG. 18A, Average number of genes 719.364806866953, Average number of reads 16860.3433476395, and Average number of UMIs 2315.01716738197. FIG. 18B Average number of genes 848.639269406393, Average number of reads 18217.0319634703, Average number of UMIs 2418.7899543379. FIG. 18C Average number of genes 798.224489795918, Average number of reads 31725.112244898, Average number of UMIs 2647.48469387755.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A method of detecting the presence of nucleic acid moiety functionalization on a solid support comprising:
contacting the solid support with a fluorescent probe, the fluorescent probe comprising an oligonucleotide, the oligonucleotide capable of binding moieties when present on the solid support.

2. The method of claim 1, further comprising quantifying the amount of functionalization of the solid support.

3. The method of claim 2, wherein the quantifying comprises measuring the level of fluorescence, the fluorescence correlating to the amount of functionalization.

4. The method of claim 1, further comprising sorting the solid support fluorescently.

5. The method of claim 4, further comprising removing the probe subsequent to sorting.

6. A method for preparing a population of functionalized solid support comprising surface reactive nucleic acid molecules in sequence(s), comprising:
reacting a solid support comprising a surface reactive nucleic acid molecule with a nucleic acid molecule so as to obtain a solid support comprising surface reactive nucleic acid molecules in sequence(s).

7. The method of claim 6, wherein reacting with the nucleic acid molecule comprises reacting with a dinucleotide or a trinucleotide.

8. The method of claim 6, wherein the reacting step is performed n times, wherein n is an integer between 1 and 150.

9. The method of claim 8, wherein the reacting step is repeated so as to obtain surface reactive nucleic acid molecules in sequences of a Universal sequence, barcode, a Unique Molecular Identifier (UMI) and a capture sequence; or so that the surface reactive nucleic acid molecules comprise one or more of the following:
oligonucleotides, nucleotides, analogs thereof;
a molecular barcode;
a Unique Molecular Identifier;
a oligodT;
an amplification primer;
a cell type specific sequence;
a pathogen-specific sequence;,
a TCR or BCR specific sequence; or
one or more primers for a specific gene, pathway, or pools of genes.

10. The method of claim 8, wherein the cell type specific sequence comprises mutation specific sequences, sequences adjacent to a feature of interest, or gene pools.

11. The method of claim 6, wherein at least one or more of the nucleic acid molecule(s) comprise a protecting group.

12. The method of claim 11,further comprising after the reacting step, deprotecting of the nucleic acid molecules is performed at room temperature in ammonia hydroxide, optionally 30% ammonia hydroxide for one hour.

13. The method of claim 6, wherein the solid support comprises a bead, micro-bead, micro-assay, micro-well, or micro-lid.

14. The method of claim 13, wherein the solid support comprises a bead that is a silica bead, a hydrogel bead or a magnetic bead.

15. The method of claim 13, wherein the solid support comprises a magnetic core.

16. The method of claim 6, wherein the solid support has an average particle size between about 10 microns to 200 microns, about 10 microns to 30 microns, about 30 microns to 50 microns, about 50 microns to 100 microns, about 100 microns to 200 microns, or about 30 microns.

17. The method of claim 6, wherein the solid support comprises a polymer, optionally a hydroxylated methacrylic polymer, a hydroxylated poly(methyl methacrylate), a polystyrene polymer, a polypropylene polymer, a polyethylene polymer agarose, or cellulose.

18. The method of claim 6, wherein the solid support comprises a hydrogel bead or a magnetic bead.

19. The method of claim 6, wherein the functionalized solid support comprises a spacer, the spacer having a functional group exposed for reaction prepared by a method comprising:
a′) reacting a solid support having surface bearing reacting groups with an activator, so as to obtain a solid support with an activated surface comprising an activating moiety; and
b′) reacting the activated surface with a spacer compound having a first moiety that reacts with the activating moiety and optionally a second moiety comprising a functional group.

20. The method of claim 17, wherein the functional group exposed for reaction comprises an amine, hydroxyl, carboxyl, or thiol.

21. The method of claim 6, wherein the solid support comprises a spacer, the spacer optionally comprising a polyethylene glycol polymer (PEG), alkyl amine, or polysaccharide linker.

22. The method of claim 21, wherein the PEG is a hetero-functional PEG comprising two or more different functionalities, wherein at least one of the functionalities is a primary amine, hydroxyl, thiol, methoxy, or other capping group.

23. The method of claim 21, wherein the PEG has a molecular weight range of about 2,000 daltons to 10,000 Daltons.

24. The method of claim 22, wherein the PEG has a molecular weight of about 2000 Daltons, 3500 Daltons, 5000 Daltons, 8000 Daltons, 9000 Daltons, or about 10,000 Daltons.

25. The method of claim 6, wherein the nucleic acid molecules comprise one or more of oligonucleotides, nucleotides, analogs thereof; a molecular barcode; a Unique Molecular Identifier; a oligodT; an amplification primer; a cell type specific sequence; a pathogen-specific sequence; a TCR or BCR specific sequence; primers for specific genes; a well barcode; primers for pools of genes; and surface reactive nucleic acid molecule(s).

26. The method of claim 20, wherein the spacer comprises a photolabile linker, a fluoride ion labile linker, or a cleavable linker.

27. The method of claim 20, wherein the spacer comprises a benzenesulfonylethyl linker, an o-nitrobenzyl carbonate photolabile linker, a 5-methoxy-2-nitrobenzyl carbonate photolabile linker, an o-nitrophenyl-1,3-propanediol base photolabile linker, a fluoride ion labile diisopropylsilyl linker, a fluoride ion labile disiloxyl phosphoramidite linker, a NPE carbonate linker, a 9-fluorenylmethyl linker, a phthaloyl linker, an oxalyl linker, a malonic acid linker, a diglycolic acid linker, a hydroquinone-O,O′-diacetic acid (Q-linker), or a thiophospate linker.

28. The method of claim 26, wherein the spacer comprises a benzenesulfonylethyl linker cleavable with triethylamine/dioxane, a nonyl phenol ethoxylate (NPE) carbonate linker cleavable with 1,8-Diazabicyclo[5.4.0]undec-7 -ene (DBU)/pyridine, or a 9-fluorenylmethyl linker or a phthaloyl linker cleavable with DBU.

29. The method of claim 20, wherein the spacer comprises a succinic acid linked to an N-methylglycine (sarcosine) derivatized support, a succinic acid linked to 1,6-bis methylaminohexane spacer, a succinic acid linked to N-propyl polyethylene glycol Tentagel support, or a succinyl-sarcosine linkage.

30. The method according to claim 6, wherein the nucleic acid molecules are in a 3′ to 5′ orientation, or wherein the nucleic acid molecules are in a 5′ to 3′ orientation.

31. The method of claim 25, comprising a plurality of solid supports, the solid supports comprising the well barcode, primers, a unique molecular identifier, and an oligo-dT or capture oligonucleotide, further comprising pooling beads in an individual discrete volume, seeding cells in the individual discrete volume, and conducting high throughput RNA sequencing for a population of cells in each individual discrete volume.

32. The method of claim 31, wherein the well barcode comprises about 6 nucleotides, the UMI comprises about 14 oligonucleotides.

33. The method of claim 31, further comprising compressive sensing for ultra-low depth sequencing.
